(19) United States
(12) Patent Application Publication
(10) Pub. No.: US 2010/0172865 A1
(43) Pub. Date: Jul. 8, 2010

(54) METHODS OF ENHANCING HAIR GROWTH

(76) Inventors: Totada R. Shantha, McDonough, GA (US); Erica Maya Shantha, McDonough, GA (US); Jessica Gowramma Shantha, McDonough, GA (US); Usha T. Shantha Martin, Macon, GA (US); Lauren Asha Shantha, McDonough, GA (US)

Correspondence Address: PATWRITE LLC, 408 W. MAIN ST., MARSHALLTOWN, IA, 50158-5759, US

(21) Appl. No.: 12/727,117

(22) Filed: Mar. 18, 2010

(51) Int. Cl. A61K 38/20; A61K 38/28; A61K 38/18; A61P 17/14

(52) U.S. Cl. 424/85.2; 514/3; 514/12

(57) ABSTRACT

A method of enhancing scalp hair growth utilizes insulin and/or IGF-1 alone or in combination with other known hair growth promoting therapeutic, pharmaceutical and biological agents to increase the therapeutic effects of hair regrowth strategies to enhance hair growth on the scalp and as method of treatment for balding.

### FIELD OF INVENTION

This invention relates to methods for stimulating the growth of human and animal hair with the application of insulin and insulin like growth factors (IGF-1) to the balding (depilation) scalp skin with or without the application of various known therapeutic, pharmaceutical, biochemical and biological hair growth accelerants.

### BACKGROUND OF THE INVENTION

Balding is a normal process that develops to some degree in all men. It can be patchy, or widespread. Dermatologists, cosmetologists and hair dressers recognize many types of hair loss, and come across human seeking longer luscious hair to add to their beauty. In woman, the hair loss involves widespread thinning rather than the development of bald patches as seen men. A gradual thinning of the hair occurs in one in eight women before menopause (the end of menstrual periods in women), and in more than one in three women after it. But it differs from the hair loss experienced in men (Balding), because complete hair loss in women is hardly ever seen. Hormones play an important role in hair loss in women as seen past middle age. The Scalp hair normally thickens and becomes more lustrous during pregnancy, but childbirth triggers the switching of active hair roots growth to the resting phase resulting in hair loss occurs in three months after delivery.

To understand the scalp hair growth, it is important to have knowledge about the hair histology, physiology and the factors contributing to their growth or loss. The hairs are found all over the skin surface of the human body. Hair is a keratinized dead cells derived from the multiplying epithelial cells in the matrix at the base of the hair follicle. Hairs are absent from the palms of the hands, the soles of the feet, the dorsal surfaces of the distal phalanges, the umbilicus, the glans penis, the inner surface of the prepuce, the inner surfaces of the clitoris, labia majora and minora, conjunctiva along and cornea. The hairs differ in length, thickness and color in different parts of the body. Hairs differ in different races of human and sex. The scalp hairs are long, can grow very long, thick and strong as compared to the hairs on the other parts of the body.

A scalp (and other hairs) hair consists of a root, the part implanted in the skin called the hair root (radix), and a shaft (scapus), the portion projecting from the surface. The root of the hair has a proximal enlargement called hair follicle embedded in the bulb, which is set in an invagination of the epidermis and superficial portion of the corium, called the hair follicle (FIGS. 1, 2). A hair follicle is a tiny cup-shaped pit buried deep in the fat (subcutaneous layer) of the scalp. The follicle is the point from which the hair grows. It is richly supplied with blood vessels and surrounded by delicate sensory nerve fibers. Blood supply is important for hair growth. The main function of blood circulation to the papilla is to transfer nutrients or other required materials such as oxygen to hair follicle and root. The blood vessels around hairs in the anagen phase are more plentiful than those around hairs in telogen phase, and it is known that as hair papilla becomes the larger, the number of micro-blood vessels increases around hair follicle. The temperature around the follicle is normal body temperature, and is not easily affected by cold or hot weather. The hair follicle can be divided into two regions.

1. The hair bulb lies inside the hair follicle. It is a structure of actively growing cells, which eventually produce the long fine cylinder of a hair.

2. The portion of hair we all desire and notice is protruding above the level of the epidermis is called the hair shaft, and the portion within the follicle buried below the epidermis is the hair root or bulb (see FIGS. 1, 2).

When the hair is of considerable length, the follicle extends deeper into the subcutaneous tissue. The longer the scalp hair, the deeper the follicle extends. The hair follicle commences on the surface of the skin with a funnel-shaped opening, and passes inwards and become dilated at its deep extremity, where it corresponds with the hair bulb which gets incorporated into hair follicle. The ducts of one or more sebaceous glands open into the follicle near the skin surface. At the bottom of each hair follicle there is a small conical vascular prominence called dermal hair papilla which is continuous with the dermal layer of the follicle, and is supplied with myelinated and non-myelinated nerve endings. The hair derives its nutrition for its growth from the capillaries in the papilla and trophic factors from the sensory nerves (FIGS. 1, 2).

Much is known about various types of human hair and its growth patterns on various parts of the body. Growth of a hair occurs at the hair bulb due to proliferation of the cells capping the papilla (stem cells) and form the germinal matrix of the hair. The duration of life of a single hair varies from about four months (for eyelashes) to about four to seven years (scalp hair, and adult beard), after which it is shed and is replaced by the sprouting of new cells from the germinal matrix (anagen phase) from the follicle after a period of rest (Telogen phase).

The hair follicle has a complex histological structure (see FIGS. 1, 2). It is composed of epithelial and connective tissue sheaths. The epithelial sheath is in close contact with the hair root and is made up of two layers; inner and outer. The inner layer is composed of three sub layers of cells: (a) an inner layer, the cuticle, which is similar and in close contact with the hair cuticle; (b) a middle Huxley's layer, made of a few rows of square cells; and (c) an outer, Henle's layer, made of one row of polygonal, flattened cells. The outer epithelial layer is considered to be nothing but down growth of epidermis, with the spinous layer inside and the basal layer and basal lamina outside. The basal lamina is thickened and known as the vitreous membrane. A connective tissue sheath is an extension of the dermis: it has two layers, inner papillary and outer reticular. Below the dense connective tissue, and external root sheath lays a glossy membrane (see the FIGS. 1, 2).

The bottom of the hair root is enlarged and made of cells with high potential for division and differentiation similar to stem cells (FIG. 1, 2). These cells comprise what is known as the hair matrix germinal cells. The hair matrix cells divide and move up the follicle, differentiating into either hair cells or inner epithelial sheath cells. Among matrix cells, there are melanocytes producing pigment of the hair (FIG. 2). The pigment is synthesized from the amino acid tyrosine (catalyzed by the enzyme phenol-oxydase) and transformed through DOPA to dopaquinon.

Further transformation of dopaquinon proceeds in two directions: either spontaneous transformation to indolquinon or through the addition of the amino acid cystein. Polymerization of indolquinon produces the dark pigment, melanin. Polymerization of indolquinon and dopaquinon with added cystein produces the yellow pigment, pheomelanin. Matrix cells during their differentiation ingest (by phagocytosis) melanin or pheomelanin from dendritic elongations of melanocytes.

This is how hair assumes its color: black if melanin is dominant, and yellow or red if pheomelanin is the major pigment. The portion of connective tissue root sheath that is in intimate contact with the hair matrix is known as the dermal papilla. It has a major regulating role in hair growth which are described Jankovic and Stenn (Slobodan M. Jankovic and Snezana V. Jankovic. The control of hair growth Dermatology Online Journal 4(1): 2, 1998, Center for clinical and experimental pharmacology Clinical Hospital Centre, Kragujevac, Serbia, Yugoslavia and K. S. Stenn and R. Paus (2001). “Controls of Hair Follicle Cycling”. Physiological Reviews 81 (1): 449-494).

There are three types of hair on the human skin besides lanugo hair seen before and shortly after birth. They are:

1. Terminal hairs (scalp, genital and beard) which are dark, thick and long;

2. Vellus hairs (rest of the body surface);

3. And modified terminal hairs such as eye lashes, and eye brows.

Terminal hairs are coarse, pigmented long in which the bulb of the hair follicle is seated deep in the dermis, hence difficult to pull out. Vellus hairs are fine, short, thin, non-pigmented or lightly pigmented hairs in which the hair bulb is located superficially in the dermis, lack medulla and easy to pluck with least pain.

As alopecia (hair loss, balding, depilation) progresses, a transition takes place in the area of approaching hair loss wherein the hairs change from the terminal or modified terminal to the vellus hair. This condition involves patchy or widespread hair loss with normal underlying skin; the cause is unknown. The function of the hair growth-promoter therapeutic agents is to replace these vellus hair with terminal hair by stimulating the growth in the hair follicle and bulb.

There are different claims on how many strands of hairs are there on an average head (scalp), but the consensus seems to be around 100,000-150,000; some estimates going up to 200,000. The scientists have found that the number of strands of hair on a human scalp varies with color of the hair: blonde—140,000 strands; brown—110,000 strands; black—108,000 strands; red—90,000 strands. It is estimated that non bald scalp contains 460 hairs compared to 306 in the bald human scalp per square centimeter. Just about 50% of the hairs must be shed to produce visible thinning of scalp hair. Passing the hairs from terminal to vellus phase, increased number of telogen hairs with loss of hair follicles that produces baldness. There is a great deal of knowledge on hair growth cycles but less is known is known about its basis. Both genetic and hormonal causes are blamed. Surprisingly, treatment with hormone modulation has been disappointing. The scalp sheds 100-150 hairs every day and hundreds of vellus hairs.

The process of balding (loss of scalp hair) is due to progressive miniaturization of individual hair follicles, which become smaller and have a shorter growth cycle. The hairs consequently become smaller and narrower. The number of hair follicles remains the same, however. There is the same number of hair follicles in the scalp of a bald man and woman as in the scalp of a man woman with a full head of hair.

Embryologic Development of Hair

In utero, the fetal epithelium of the skin (ectoderm) and underlying mesenchyme interact to form hair follicles, from which hair shaft protrudes. During the third month of intrauterine gestational life, the epithelial primary hair germinal cells develop in the scalp and eye brows. Subsequently, hair development continues caudally in a head to toe direction. The primary hair germ begins as an epithelial bud on the epidermis that protrudes into the dermis and seems to be directed by a group of mesenchymal cells that eventually form the dermal (follicular) papilla. The germ cells continue to grow and spread deeper in the dermis under the guidance of the dermal papilla. It then acquires multiple epithelial buds that grow horizontally and differentiate into the many components of the primary hair follicle such as sebaceous gland and duct, apocrine gland and duct, attachment point of the arrector pili muscle of the hair root bulge region. Sebaceous glands are absent in the palms and soles; apocrine glands develop only in certain body regions. Melanocytes are scattered haphazardly in the primary epithelial bud which contribute the hair color.

Hair Growth

In humans, hairs grow in cycles which are not synchronized. Each hair undergoes 4 phases of the growth cycle at a different time namely: anagen, catagen and telogen (K. S. Stenn and R. Paus (2001). “Controls of Hair Follicle Cycling”. Physiological Reviews 81 (1): 449-494). Anagen is the phase of active hair growth—approximately 90% of all hairs are in anagen lasting 2 to 7 years, depending on skin region. After anagen is completed, the hair enters catagen; during this short phase (2-3 weeks) the matrix cells gradually stop dividing and eventually keratinize.

When full keratinization is achieved, the hair enters the last phase of the cycle, telogen phase lasting 3-4 months. The keratinized hair falls out. After this telogen phase, new matrix is gradually formed from stem cells in basal layer of outer epithelial root sheath bulge and germinal cells surrounding the papilla (FIGS. 1, 2). A new hair starts to grow and the follicle is back in anagen phase. In summary, the hairs undergo the following growth and shedding cycles:

Anagen—active growth—new hair is pushing out the old fiber and the follicle is growing deep for nourishment

Catagen—the transitional phase. Hair detaches from the blood supply and the hair follicle shrinks

Telogen—Resting phase. Hair fiber easily pulls out

Mesanagen—a returning to growth pattern

Telogen Effluvium Hair Loss: It is a condition when some stress or infection or drug causes hair roots to be pushed prematurely into the resting state. Telogen effluvium can be acute or chronic. There is shock to the hair root resulting as many as 70% of the scalp hairs are shed in large numbers about 2 months after the “shock”. In acute telogen effluvium, a handful of hair comes out easily when pulled. High fevers, childbirth, severe infections, severe chronic illness, severe psychological stress, loss of loved ones, major surgery, prolonged major illnesses, over or under active thyroid gland, crash diets with less protein, massive radiation and chemotherapy and a variety of medications such as retinoids, beta blockers, calcium channel blockers, antidepressants, and NSAIDS (including ibuprofen). Hair growth returns slowly when the offending agent is removed and may need to be treated using our inventive method with or without approved hair growth therapeutic agents. The latest studies show the stress based biological elucidation of how stress may trigger or aggravate telogen effluvium and alopecia leading to baldness. (Eva M. J. Peters,* Sofia Liotiri,† Enik [symbol] Bodó,† Evelin Hagen,‡ Tamás Bíró,§ Petra C. Arck,‡ and Ralf Paus† Probing the Effects of Stress Mediators on the Human Hair Follicle Substance P Holds Central Position Am J Pathol. 2007 December; 171(6): 1872-1886)

Exogen: It is a term used for shedding of the hair in mammas. The shedding phase occurs independent of telogen and anagen, it probably utilizes a separate set of controls; for this reason, the shedding phase has been given a distinct name, Exogen (Stenn K S, Parimoo S, and Prouty S. Growth of the hair follicle: a cycling and regenerating biological system. In: Molecular Basis of Epithelial Appendage Morphogenesis, edited by Chuong C-M. Austin, Tex.: Landes, 1998, p. 111-130.), in line with the anagen, catagen, and telogen phase names coined by Dry (Dry F W. The coat of the mouse (Mus musculus). J Genet. 165: 281-340, 1926). Little attention has been given to the mechanism of hair shedding, although it would appear that shedding is probably the most important aspect of hair growth from a patient's perspective. In animals a new hairs grow before old ones fall out. This is a protective mechanism, which minimizes the possibility of shedding the protective fur before new fur is available. For example, Rats and mice, have two and often three hair shafts in a follicle at the same time while only one is growing. In sheep, and many animals, it influenced by environment (light, temperature, and nutrition) and systemic (endocrinological factors) parameters, it is generally believed that there is considerable innate local control of hair shedding; each fiber grows to a specific length, for a specific period of time, before being shed (Paus R, Muller-Rover S, and Botchkarev V A. Chronobiology of the hair follicle: hunting the “hair cycle clock.”J Invest Dermatol Symp Proc 4: 338-345, 1999). The details on control of hair follicle cycling are discussed in a exhaustive review article by K. S. Stenn and R. Paus; Controls of Hair Follicle Cycling, Physiol. Rev. 81: 449-494, No. 1, January 2001.

What initiates anagen from telogen in the mature follicle and from where that stimulus comes is not established? The signal could arise in the resting papilla, the resting epithelial germ and stem cells, the adjacent epidermis, even from the supportive vessels, nerves, lymphatics, and resident dermal hematopoietic cells of the region. Tissue culture studies suggest that the signal arises independent of neural elements and vascular or endocrine signals.

The testosterone, an androgenic hormone stimulates hair growth when applied topically to the deltoid area and injected into the beard and pubic regions but not on the scalp. Oral administration result in an increased hair growth in the beard, pubic region, on the trunk and extremities. It is ineffective when applied topically to the scalp balding area and may even cause more thinning of the hair.

Androgens have diverse effects on hair growth in different body regions (Randall V A, Thorton M J, Hamada K, Messenger A G. Androgen action in cultured dermal papilla cells from human hair follicles. Skin Pharmacol 1994; 7: 20-6). Effects vary from essentially nonexistent (e.g. on eye lashes), weak (on temporal and sub occipital region hair), moderate (on extremity hair), or strong (on facial, parietal region, pubic, chest, and axillary hair). Androgens bind to receptors both in the cytoplasm and nuclei of dermal papilla cells and stem cells of the sheaths of the follicle, but only if the hair is in anagen or telogen phase (Sawaya M E. Purification of androgen receptors in human sebocytes and hair. J Invest Dermatol 1992; 98(6 Suppl):925-965. Chondrhry R, Hodgins M B, Van-der Kwast T H, et al. Localization of androgen receptors in human skin by immunohistochemistry: implications for the hormonal regulation of hair growth, sebaceous glands and sweat glands. J Endocrinol 1992; 133: 467-75.).

Among all androgens, dermal papilla cells are most affected by 5α-dihydrotestosterone (5α-DHT). It is synthesized in these cells from testosterone under catalytic action of the enzyme 5 α-reductase (Randall V A. Role of 5α-reductase in health and disease. Bailliers Clin Endocrinol Metab 1994; 8: 405-31). This enzyme exists in two forms (isoenzymes)—type I and type II (Horton R. Dihydrotestosterone is a peripheral paracrine hormone. J Androl 1992; 13: 23-7). 5α-dihydrotestosterone is further reduced to 3α-androstanediol which, after conjugation with glucuronic acid, is excreted in urine. Plasma and urine levels of 3α-androstanediol glucuronide are the precise clinical indicators of the extent of testosterone transformation to 5α-DHT (Toscano V, Balducci R, Bianchi P. et al. Two different pathogenetic mechanisms may play a role in acne and in hirsutism. Clin Endocrinol Oxf 1993; 39: 551-6). They are elevated in hirsute women.

Another important enzyme that plays a role in hair loss is Aromatase, which present in the skin fibroblasts and absent in the dermal papilla. Aromatase activity has been detected in plucked human scalp hair follicles (Schweikert H U, Milewich L, Wilson J D: Aromatization of androstenedione by isolated human hairs. J Clin Endocrinol Mtab 40: 413, 1975. Sawawa M E, Penneys N S: Immunohistochemical distribution of aromatase and 3B-hydroxysteroid dehydrogenase in human hair follicle and sebaceous gland. J Cutan Path01 19: 309, 1991). In hair follicles in most body sites, testosterone acts as a pre-hormone and the response to testosterone is determined by which metabolic pathway predominates as disclosed in U.S. Pat. No. 6,020,327.

In skin sites where 5α-reductase activity is high, such as in the beard, testosterone is metabolized to DHT which has a stimulatory action on hair growth on the rest of the body except the scalp. In scalp, testosterone is metabolized mainly via the aromatase pathway to estrogens causing inhibition of hair growth, hence balding and scalp hair thinning. Aromatase activity is under receptor-mediated androgenic control; hence balding does not occur in testicular feminization.

The greater severity of balding in men compared with women can be explained by higher levels of estrogens being formed in the male scalp due to androgen stimulated aromatase activity. It is known that the estrogens inhibit the hair growth in mammals (Moho M P: The effects of different hormonal states on the growth of hair in rats, in Montagna W, Ellis R A (eds): The Biology of Hair Growth, New York, Academic Press, pp 335-398, 1958).

An analogous situation exists in the brain where some uniqueness of male behaviors in a number of avian and mammalian species are determined by estrogens synthesized locally by androgen-dependent aromatization of androgens (Hutchinson J B: Aromatase: neuromodulator in the control of behavior. Steroid Biochem Molec Biol 44: 509, 1993.). This hypothesis is also able to encompass a role for 5α-rcductasc; as DHT is a more potent androgen than testosterone, circulating or locally formed DHT can be a more effective inducer of aromatase.

Factors Influencing Hair Growth

The hair follicle is a regenerating system and the most important function of the hair follicle is to produce a hair shaft (hair fiber). By passing through the phases of the cycle (growth, regression, resting, shedding, then growth again), the follicle demonstrates the unusual ability to completely regenerate itself due to uniqueness of follicular epithelial and mesenchymal components and their interactions. The follicles are different from site to site of different scalps producing hair shafts differing in size, shape, curl, and color.

Hair follicles contain abundant Stem cells in interspersed within the basal layer of the outer root sheath and in an area called the hair bulb bulge adjacent to the papillary projections. It is from these cells that matrix cells are formed (FIGS. 1, 2). Growth and differentiation of the matrix contribute to the hair growth and these cells are under the influence of substances produced by cells of the dermal papilla. On the other hand, the secretory activity of the dermal papilla is controlled either by substances produced in cells of the spinous layer of the outer root sheath and/or by hormones. Cells of the spinous layer produce peptides greater than 3000 Daltons which increase the number of papilla cells mitosis two to five times. It was recently discovered that basic fibroblast growth factor (bFGF) and platelet-derived growth factor (PDGF) potentiate the growth of dermal papilla cells. It is proposed that these proteins increase the synthesis of stromelysin (an enzyme, matrix metalloproteinase—MMP) which acts on the papilla cells and accelerates their growth. Another cytokine, transforming growth factor beta (TGF- [symbol] ), inhibits mitogen-induced dermal papilla cell proliferation. On the other hand, dermal papilla cells produce several cytokines which influence proliferation of hair matrix cells hence influence the hair growth. Some of them are stimulators, and some inhibitors. Interleukin 1- [symbol] (IL-1^{u}) inhibits growth of hair and follicle, but only after 2-4 days of latency. The increase of IL-1 [symbol] concentration in extracellular fluid during inflammation could be one of the reasons for alopecia following certain infectious diseases and septic shock besides decreased blood supply and inhibition by bacterial toxins. Apart from IL-1α, both fibroblast growth factor (FGF) and epidermal growth factor (EGF) inhibit growth of the hair and hair follicle. Fibroblast growth factor type 5 (FGF5) is an especially potent inhibitor (Harmon C S, Nevins T D. IL-1 α inhibits human hair follicle growth and hair fiber production in whole organ cultures. Lymphokine Cytokine Res 1993; 12: 197-203.). Receptors for these ligands were found by immunohistochemical methods on papilla cells, matrix cells and stem cells in the bulge region (see FIGS. 1, 2) of the hair follicle (Du Cros D L. Fibroblast growth factor and epidermal growth factor in hair development. J Invest Dermatol 1993; 101(1 Suppl): 106S-113S. Akiyama M, Smith L T, Holbrook K A. Growth factor and growth factor receptor localization in the hair follicle bulge and associated tissue in human fetus. J Invest Dermatol 1996; 106: 391-6). Another cytokine produced by cells of the dermal papilla called keratinocyte growth factor (KGF), induces extensive hair growth in murine models of alopecia. Receptors for KGF were found on keratinocytes in the basal epidermis and throughout developing hair follicles of rat embryos and neonates (Danilenko D M, Ring B D, Yanagihara D, et al. Keratinocyte growth factor is an important endogenous mediator of hair follicle growth, development, and differentiation. Normalization of the nu/nu follicular differentiation defect and amelioration of chemotherapy-induced alopecia. Am J Pathol 1995; 147: 145-54).

Dexamethasone stimulated aromatase 10-20 fold in fibroblasts and induced aromatase in dermal papilla cell. The high levels of aromatase in fibroblasts from frontal scalp are consistent with hypothesis that it plays a role in frontal male baldness. The recognized inhibitory effect of estrogens on hair growth would need to derive from the surrounding dermis as aromatase activity is absent in dermal papilla cells. There is no evidence that androgens act directly on the hair follicle in the balding process. The alteration in skin texture of subjects of a balding scalp suggests that there is an alteration the skin structure which may play a role in the hair loss or hair growth inhibition besides the reduced blood supply to the area of balding. Hence, it is likely that the inhibition of hair growth is a secondary response to more general changes in local biology of the skin.

U.S. Pat. No. 6,020,327 discloses many factors involved in the hair growth including aromatase inhibitors which are incorporated in the following description. There is great evidence that androgen hormones are necessary for the development of balding; though the mode of hormone action on the hair follicle is unspecified. At present there is no satisfactory explanation and treatment for scalp hair loss (balding). Beard and pubic hair need androgens to prolong the anagen phase for hair growth as exemplified by the appearance of the beard with elevated testosterones in male teen agers. Scalp hair does not need androgens for hair growth but are needed for balding.

It is important to note that there is no difference between the balding and non balding male in their plasma concentration to testosterone. It is thought that the in the skin sites where 5α-reductase activity is high, such as in the beard, testosterone is metabolized to DHT which has a stimulating action on hair growth. On the other hand, in the scalp, the testosterone is metabolized mainly via the aromatase pathway to estrogens causing inhibition of hair growth hence balding effect.

The greater balding in men compared with women is explained by higher levels of estrogen being attained in the male scalp by virtue of androgen stimulated aromatase activity pathway. The formed DHT can be a more effective inducer of aromatase cycle as DHT is a more potent androgen than testosterone. The circulation or locally formed DHT can be a more effective inducer of aromatase resulting is female hormone formation resulting in male pattern baldness. Administration of 5α-reductase inhibitors and Aromatase inhibitors in macaque's monkeys which undergoes androgen dependant scalp hair loss like human prevents and reverses the hair loss. Men with type II 5α reductase deficiency do not go bald either supporting the hypothesis it plays in converting testosterone to DHT which stimulates the aromatase inhibitors to form estrogens. This is another indication of the important role played by 5α-reductase and aromatase in balding of male.

Causes Hair Loss

Scalp hair loss and thinning (depilation, age related thinning, alopecia etc) may not be the most common form of hair loss, but it can be a stressful in those who are appearance and beauty-conscious. Any acute, chronic systemic and local scalp disease can result in loss of hair. The biggest culprit is the treatment of cancer with use of massive doses of chemotherapy and radiation which inhibits or kills the multiplying germ cells of the hair bulb and follicle. They do recover, and many of the hair follicles grow back, reseeding the scalp with normal hair.

The following conditions can result in hair thinning and/or loss:

Aging is one of the commonest causes of scalp hair thinning or hair loss due to hormonal effect, reduced blood supply and various age related changes of the scalp and its hair.

Alopecia areata is a medical condition where body's immune system starts attacking hair follicles which will affect hair on the entire body, including the scalp hair.

Hair root infection of the scalp hair including dandruff can attack hair root resulting in swelling and itching due to excessive bacteria growth in tiny oil glands resulting some hair loss. This will not cause general hair loss.

Allergic reaction to the various scalp makeup products can affect the hair growth due to various anti hair growth cytokine production and effect on the germinal cells due to allergic reaction.

Demodex folliculorum is a type of mite that exists naturally in skin pores and hair follicles. These mites cause scalp hair to fall out.

General infection caused by a variety of reasons can swell the hair bulb and hair loss. Septic shock and extreme stress can result in scalp hair loss.

Hyper or Hypothyroidism: overactive or underactive thyroid can cause areas of hair to thin, become brittle and fall out.

Improper care such as using harsh cosmetics or forgetting to remove makeup before bed can cause hair damage. They can become brittle and thus break or fall out.

Poor diet is always a cause for general hair thinning or hair loss such as seen in serious bulimia (After prolonged vitamin deficiencies, or a deficiency of protein or iron), which wrecks the body hair including scalp.

Trauma to the scalp can result is loss of hair including ripping the scalp, and burning of the scalp.

Medications are a frequent cause of hair loss including some heart and blood pressure medications, thyroid medications, cholesterol lowering drugs etc.

Radiation—accidental or therapeutic results in hair loss. Radiation affects the nuclear DNA needed for cell growth and multiplication at the papilla and bulb. Without multiplication of germinal cells, hair cannot grow, hence hair thinning and loss.

The following drugs, medications, substances or toxins (toxic substances such as thallium (thallium sulfate is widely used as a rat poison) may possibly cause scalp hair loss such as Acitretin (Soriatane, Neotigason), chemotherapeutic agents, corticosteroids, estrogens etc.

Ringworm: Small patches of hair loss associated with dandruff-like scaling of the scalp and short stubby, broken hair shafts within the bald area is typical of scalp ringworm. It occurs in children and is contagious, but it is not a serious threat to health. Once the diagnosis is made, the condition is easily treated with anti-ringworm treatment with return of hair growth.

Alopecia greata: This condition entails patchy hair loss with normal underlying skin with no known cause. As the hair in one patch starts to grow again, another bald patch may start developing. No cure for this condition is known, but it usually clears up by itself in six months to a year and can be effectively treated by our invention.

Other causes of hair loss: Ichthyosiform erythroderma (nonbullous congenital, Ichthyosis), Keratosis Follicularis Spinulosa Decalvans, Leprosy, Progeria, Siemens syndrome, Spastic paraplegia—neuropathy—poikiloderma, anorexia nervosa, chronic anorexics, mental patients, diseases affection general nutritional intake, incarceration of prisoners or sect of people away from the public, holding hostages under threat and isolation etc.

Ways to Restore Hair Loss:

There is no single method which can restore the hair completely. There are various methods used by those who are conscious about their hair and their loss. Generalized hair loss—due to certain illnesses and infections, malnutrition, vitamin deficiencies, as a side effect of medication or medical treatment, and following exposure to toxic substances—is usually temporary. Because these processes do not damage the hair roots, hair will regrow in the most of people. Patchy hair loss caused by ringworm is easily reversed with anti-ringworm treatment. Alopecia greata cannot be cured, although the condition usually clears by itself within six months to a year. Our invention helps both these conditions.

Balding in men is normal with aging and those comfortable with the appearance no treatment is needed. Treatments for this condition are designed in this invention to reverse the normal and natural course of events. First diagnose the underlying etiology if there is any and treat the condition before resorting hair growth therapeutic measures described in this invention.

The following are some of the method to restore hair loss. They include:

Camouflage techniques Toupees and Hairpieces, Plastic surgery techniques and Drug treatments. Toupees and hairpieces may be made of artificial fiber or natural hair. Hairpieces often look unnatural. Hair weaving technique allows artificial or natural hair to be anchored onto, or braided into, the remaining natural hair. The artificial or natural hair tufts are sewn, woven, knitted, or glued; the anchoring needs to be tightened every six to eight weeks and washing may loosen the new hair.

Plastic Surgery Hair implantation techniques include: Plugs of bald scalp are removed and then filled with plugs of scalp containing several hair roots taken from hair-bearing areas—mostly from the back of the head. The hair follicles eventually take root and grow in to hair. Scalp flap transfer: A long thin “flap” of hair-bearing scalp is removed and replaced across a bald patch to create a dense band of normal hair growth with removal of the bald scalp. Skin expansion: stretches out the hairy scalp by inflatable bags to a greatly increased area. At a second operation, the bald scalp can be removed and the expanded hair scalp can be stretched to take its place. Serial excision: as much of the bald area as possible is surgically removed in the first operation. The adjacent hair-bearing areas of scalp are pulled close to the bald area. The surgical procedures are expensive, painful and fraught with fear of repeated procedures needed to restore the scalp hair.

Drug treatment to enhance the hair growth:

Three medications; Minoxidil (Rogaine), Finasteride (Propecia, Proscar) and Latisse have been approved for the treatment of hair loss and/or as growth accelerants by FDA. They can be used as prophylactic to avert hair loss or to increase the hair growth.

Minoxidil is applied topically, and is available without a prescription. Side effects of minoxidil though rare include: Itching or skin rash on the scalp, Headaches, Dizzy spells, Irregular heartbeat or chest pains, Decreased sexual ability or desire. Minoxidil may help hair to grow in 10% to 20% of the population, and in 90% it may slow the loss of hair. Finasteride (Propecia, Proscar): is another drug approved to be taken by mouth. It is primarily used to treat urinary problems cause by enlargement of the prostate in men (benign prostatic hyperplasia, or BPH). Finasteride has been found to stimulate hair growth in some men. Women, however, should not take finasteride. Needs a doctor's prescription. In clinical studies, it stopped hair loss in 83% of men. Side effects of finasteride include: Breast enlargement (gynecomastia) and tenderness, Skin rash, swelling of lips, abdominal pain, Back pain, Decreased sex drive & semen, even impotence, Diarrhea, Dizziness and headaches. The latest drug approved to grow eye lash hair is marketed under the name of Latisse, a prostaglandin derivative. It is not approved for balding, and there is no reason why it should not be used, except, it is prohibitively expensive for such use.

Despite manufacturers' claims, protein and collagen contained in hair sprays and shampoos cannot be incorporated into the hair shaft to enhance hair strength—although by leaving a coating on the hair, they may temporarily make hairs look thicker and is not due to hair growth.

Methods of Prevention of hair loss include:

Eating nutritious food rich in proteins, vitamins, antioxidants, and minerals. Avoid stress completely. Get treatment for any hair root infection including dandruff.

Avoiding hair treatments that can damage hair; this includes bleaching, hot rollers, frequent perming, hot curling irons, and repeated brushing and combing under hot hair dryers. Avoid vigorous combing and brushing the hair when it is wet, because its structure is more fragile then. Shun brisk rubbing, and very hot hair dryers. Brush hair with wide-toothed combs and brushes with smooth tips.

Stay away from hairstyles that put tension on hair, such as ponytails and braids, to avoid a constant pull that can pull hair out of their roots and hence hair loss. May use rogaine and propecia as the hair loss becomes apparent and bothersome to prevent further loss as prophylactic and preventive measure

The following patents disclose some inventive methods for hair growth.

U.S. Pat. Nos. 4,139,619 and 4,968,812 disclose topical formulations for hair growth using compound minoxidil for treatment of male pattern baldness. Topical application of Minoxidil produces mild to modest hair restoration effect (hypertrichotic effect) in early balding in less than 50% of the user. 2% and 5% formulations are available over the counter without prescription, and can be used in both sexes. This hair growth effect appears to involve opening of potassium channels (vasodilatation). If effective, it is a lifetime commitment. If regular application of minoxidil is halted, all results of therapy will be rapidly lost within 6 months.

There are other potassium channel agonists such as diazoxide, nicorandil which also stimulate hair growth. Diphenylhydantoin used as an anticonvulsant drug enhances the hair growth. Once the patient ceases taking the drug, the hair reverts to whatever is normal for the particular site after six months to a year has elapsed. Streptomycin is another drug that has been found to produce hypertrichosis, in much the same way as diphenylhydantoin. The treatments of balding hair with a microemulsion cream containing both estradiol and oxandrolone as its active ingredients and the use of organic silicon does cause hair growth.

U.S. Pat. No. 6,020,327 discloses many factors involved in the hair growth including aromatase inhibitors which are incorporated as whole in the following description. They disclose the method of treating hair loss by topically administering an aromatase inhibitor such as Aminoglutethimide, 4-OH androstenedione, 6-hydroximino-andmstenedione, 9,10-dimethyl 1,2-benzanthraccne 1,4,6-androstatrien-3,17-dione, Atamestane, Exemstane, Fadrozolc, Vorozole, 1,2,4-triazole-3-alamine, Rogletimide etc. Aromatase inhibitors are used in treatment of breast cancers which stops the production of estrogen in post-menopausal women by blocking the enzyme aromatase. There are: Arimidex (chemical name: anastrozole), Aromasin (chemical name: exemestane), Femara (chemical name: letrozole) used for breast cancer treatment.

U.S. Pat. No. 5,981,543 discloses the used of finasteride (Propecia) to treat male baldness. This therapeutic agent is used to treat prostate hypertrophy. It acts by reducing the enzyme 5 α-reductase; an enzyme responsible for the formation of dihydrotestosterone (DHT) from hormone testosterone.

U.S. Patent Application Publication Number: 2003/0199590 A1: disclose Methods and compositions for the promotion of hair growth in mammals, comprising PGF two alpha, analogues are disclosed.

U.S. Pat. No. 6,262,105 discloses prostaglandins and derivatives are useful in a method of enhancing hair growth. Bimatoprost, which is sold by Allergan, Inc. of Irvine, Calif., U.S.A. as Lumigan® ophthalmic solution, for treating glaucoma now has been found as being effective to increase the growth of hair and eyelashes when applied and has been FDA approved U.S. Pat. No. 6,020,327; United States Patent Application Publication No.: US 200710282006 A1 and US 200810070988 A1. These inventions relates to a method for stimulating the growth of mammalian hair comprising the application to mammalian skin of a cyclopentane heptanoic acid, 2-cycloalkyl or arylalkyl derivative or a pharmacologically acceptable acid addition salt thereof, alone, or in association with a topical pharmaceutical carrier.

U.S. Patent Application Publication Number: 200910018204 Al, discloses composition for stimulating the growth and coloration of human hair a prostaglandin or prostaglandin-like compound. U.S. Pat. No. 6,262,105 to Johnstone suggests that prostaglandins and derivatives thereof are useful in a method of enhancing hair growth. Bimatoprost, which is sold by Allergan, Inc. of Irvine, Calif., U.S.A. as Lumigan B ophthalmic solution, for treating glaucoma now has been found as being effective to increase the growth of eyelashes when applied in the FDA approved manner.

United States Patent Application Publication No.: 200310147823 Al and U.S. Pat. No. 6,020,327 disclose Methods and compositions for stimulating the growth of hair are disclosed wherein said compositions include a cyclopentane heptanoic acid, 2-cycloalkyl or arylalkyl compounds.

U.S. PATENT APPLICATION PUB. NO.: 200810145331 A1 disclose the compositions and methods for inducing hair growth and improving hair quality utilizing extracts of Cotinus coggygria in an amount effective to induce hair growth when applied topically to an area of the skin on which hair growth is desired.

U.S. Patent Application Publication Number: 200610265028 A1 invention relates to the use of photodynamic therapy (PDT) to stimulate hair growth. In particular, this invention relates to the use of photosensitizers and PDT for treating conditions relating to hair loss, such as androgenetic alopecia and alopecia greata, is described. The present invention further relates to a method of photodynamic therapy that causes an increase in the level of pro-inflammatory cytokines thereby causing hair growth and to a method of determining the increase in hair growth.

U.S. Patent Application Publication Number: 00410009135 Al, relates to a hair growth formulation comprising tocotrienol for promoting hair growth.

U.S. Patent Application Publication Number: 200910104295 A1 invention found that the processed fermented semi-mature soybean significantly enhanced hair growth compared to the raw or fully matured form.

U.S. Pat. No. 7,462,604 B2 disclose a hair growth promoter which is an ethanol or aqueous ethanol preparation comprising, as active ingredients for a hair growth promoter, (A) at least one compound selected from fatty acids having a chain length of an odd number of carbon atoms, the derivatives of the fatty acids, aliphatic alcohols having a chain length of an odd number of carbon atoms and the derivatives of the aliphatic alcohols and (B) at least one selected from 6-benzylaminopurine and/the derivatives.

U.S. Pat. No. 6,773,881 B2 discloses methods of modulating hair growth and/or hair thickness. The method includes modulating VEGF activity, e.g., modulating VEGF gene expression and/or modulating VEGF protein production and/or activity, to modulate hair growth and/or thickness. The methods can be used to either promote or inhibit hair growth or hair thickness in a subject.

U.S. Pat. No. 7,081,258 B2 invention relates to a composition for promoting hair growth by inhibiting 5a-reductase activity for promoting hair growth, comprising: a) Sophora flavescens extract; b) hiiokitiol; and c) nicotinamide, wherein the composition.

U.S. Pat. No. 7,081,258 B2 invention discloses component for inhibiting 5a-reductase activity, for promoting hair growth by promoting cell activity, and by dilating the blood vessels. The ingredient for inhibiting 5 a-reductase activity includes Sophora Flavescens extract, Coicis semen extract, clove extract, etc., and Sophora Flavescens extract is particularly preferable. Representative ingredients its root contained in Sophora fljavescens include alkaloids such as matrine, oxymatrine, sophoranol, etc.; and flavonoids such as kuraridin, isoanhydroicaritin, etc.; its leaves contain luteolin-7-glucose; and its seed contains cytosine, etc.

U.S. Patent Application Publication Number: 2005/0142088 A1 invention discloses a compound having a cholinesterase inhibiting action has a hair growth promoting action such as donepezil hydrochloride applied locally as liquid preparation, a cream, an ointment, a plaster or a tape preparation.

U.S. Patent Application Publication Number: 200310147831 Al, disclose a method of hair roots of actively growing hairs embedded in a layer of fat cells (adipocytes). The inventors noted that, while balding areas of the scalp are generally depleted of fat seldom occurs) contains a thick layer of fat tissue. They considered it likely that fat cells produce a growth factor that is essential for hair growth. They also disclose the use of component of a composition (e.g., a fluid, gel, or solid composition) also containing hair follicle cells, e.g., dermal papillae cells, outer and inner root shaft cells such as keratinocytes and fibroblasts. Such compositions can be injected into balding areas (e.g., scalp) of a patient. To obtain follicle cells, 3 to 6 mm punch biopsies of skin obtained from the occipital area of the same subject (or another subject), where there is healthy hair growth, and individual healthy hair follicles can be isolated from them. From the isolated hair follicles, their cellular components can be obtained and grown in vitro. Follicle cells include dermal papilla cells, outer shaft epithelial cells, and inner root fibroblastic cells as well. Inner and outer shaft cells can be isolated from the hair follicles. Alternatively, skin keratinocytes and skin fibroblasts obtained from a skin biopsy can be used. Such cells are known to adapt to new environments. Generally the cells to be injected will not be of one type only. Preferably the compositions will contain cells of all three types. This process could be carried out in a doctor's office without the recovery time of in-patient surgery and the patient could go back to work the same day according to the inventors. This method is not practical to be used by day to day application and is of experimental interest only at the present time. This method do not incorporated the therapeutic agent uptake, increase metabolic activity and enhance the mitosis of the adipocytes, stem cells and germinal cells responsible for hair growth as described in the present invention.

Preliminary trials indicate that Cyproterone Acetate (CPA); an androgen receptor blocker with progestational activity has positive affect in female hair loss. It has feminizing effect on men when taken orally hence cannot be used for male baldness. Topical formulations of this compound is not yet available to be used in men without the systemic effects. It has now been discovered that particular PGF 2α analogues are surprisingly effective agents for the promotion of hair growth when topically applied to man and mammals. When combined with our invention, hair growth is rapid with less prostaglandin.

The above inventions do not describe the use of insulin and IGF-1 or any such activator of stem cells and germinal matrix cells mitosis and their metabolic activity which is important for hair growth. None of these patents describe the enhancer of therapeutic agent uptake, and enhancer of their activity as described in the present invention.

### SUMMARY OF THE INVENTION

The object of the present invention if provide a hair growth stimulant for treating balding and enhancing scalp hair growth utilizing insulin and IFG-1. It also discloses methods to increase the hair growth by using therapeutic, pharmaceutical, biochemical and biological agents in combination with insulin to enhance scalp hair growth.

### BRIEF DESCRIPTION OF THE DRAWINGS

FIG. 1 shows the detailed histology of the hair follicle, hair bulb and hair shaft and their contribution to hair growth along with stem cells, germinal cells of the matrix which contributes to the healthy hair growth.

FIG. 2 is a diagrammatic presentation of the terminal hair on the scalp showing the components of hair starting from hair papilla, stem cells, germinal cells, matrix cells, hair bulb, hair root (on which insulin and IGF-1 acts to promote hair growth) and hair shaft.

### DETAILED DESCRIPTION OF THE INVENTION

In the following detailed description of the invention, reference is made to the drawings in which reference numerals refer to like elements, and which are intended to show by way of illustration specific embodiments in which the invention may be practiced. It is understood that other embodiments may be utilized and that structural changes may be made without departing from the scope and spirit of the invention.

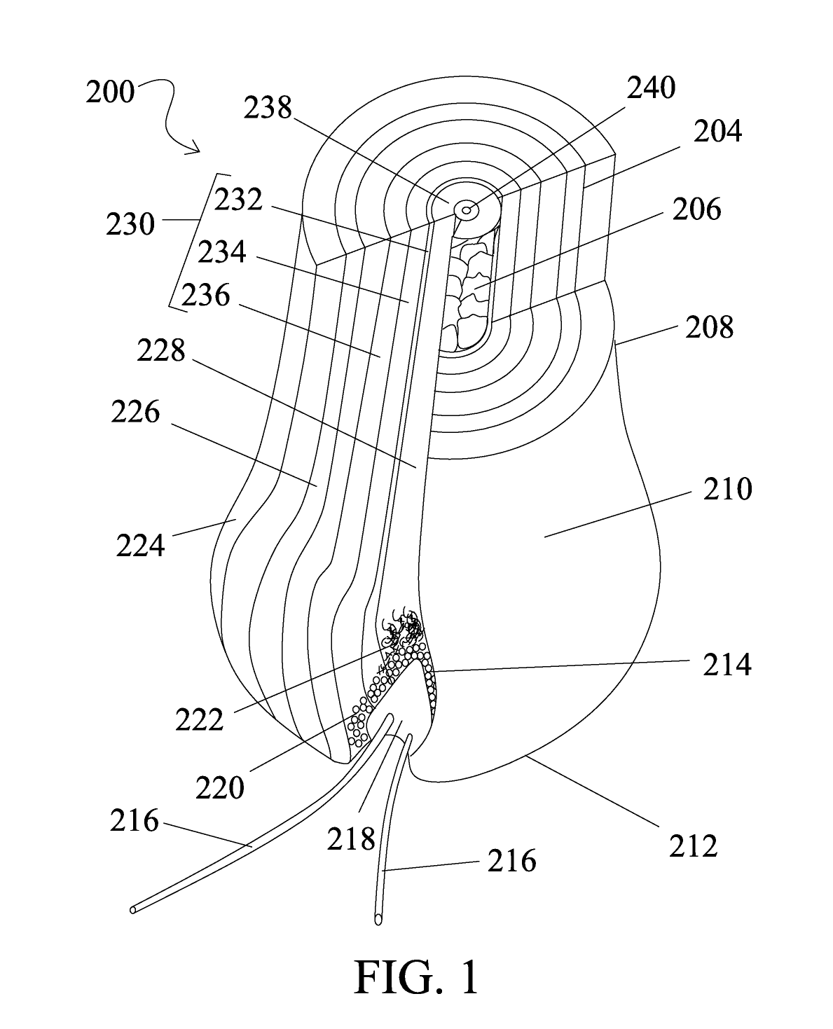

FIG. 1 is a diagrammatic presentation of the structure of the hair follicle 200, hair bulb 210 embedded in the follicle 212, hair root 208, and hair shaft. Note the location of the stem cells, matrix germinal cells which contribute to the hair growth, and the location of melanocytes responsible for hair color. Also shown are glossy membrane 204, germinal cells 214, papillary blood vessel 216, dermal papilla 218, dividing stem cells 220, melanocytes 222, dermis 224 connective tissue, external root sheath 226, zone of keratinization 228, inner root sheath 230, cuticle of inner root sheath 232, Huxley's layer 234, Henle's layer 236, 238 cortex and medulla 240.

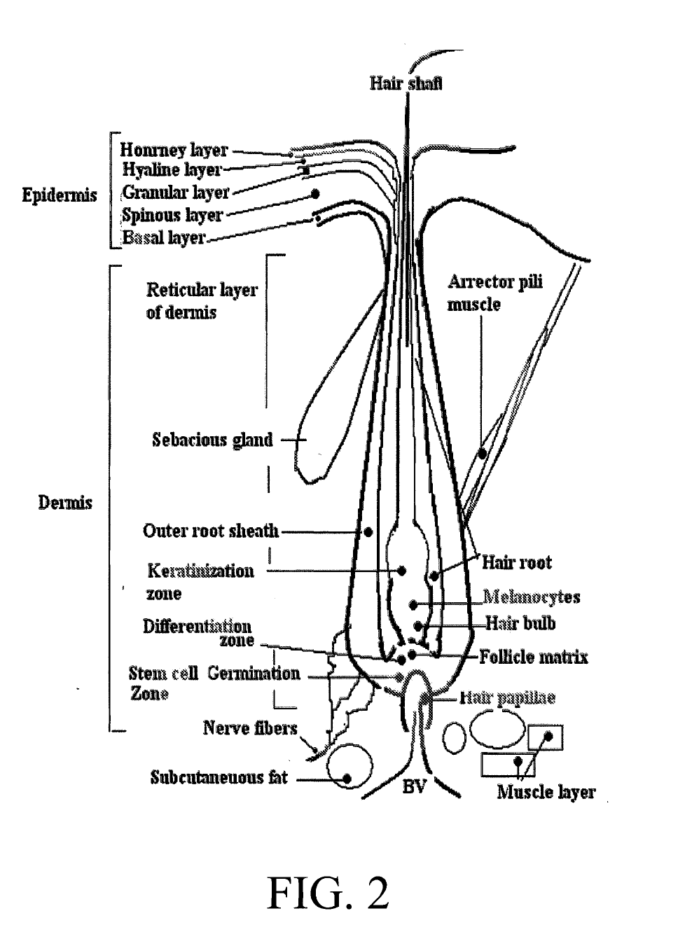

FIG. 2: Vertical section of the hair showing various components of the hair as described in the FIG. 1 to show where our invention acts to promote the hair growth. The space between the hair shaft and dermal layer permits the insulin to permeate to the hair bulb to papilla and stimulate the hair progenitor cells to multiply and contribute to the health and growth of the hair.

Scalp hair loss (Alopecia, baldness, hair thinning, depilation) is a shortage or paucity of normal terminal hair on the head. It is a deficiency of terminal hair, their broad diameter (thickness) and the colored hair that is not readily seen in this condition. Although there is a noticeable absence of terminal hair, the scalp skin does contain vellus hair which is fine colorless hair which may require magnifier to find them in the scalp. The vellus hair in the balding scalp has the capacity to rejuvenate with proper therapy as described in this invention. Along with balding scalp; due to any number of above described reasons, hair loss can be patchy (alopecia) or diffuse extending over the frontal-parietal-occipital area. This condition can be very distressing to cosmetically conscious male and females. It is one thing to have a hair growth stimulator, but it is another part to get it to the site of hair growth, i.e. hair follicle and hair bulb (see FIGS. 1, 2). We describe such agents in this invention and their site of application.

Besides aspirin and antibiotics, insulin is the most commonly used therapeutic agent known to the public and medical professional. It has been used in home by the diabetic patients or in the office by the physicians. It can be easily obtained by prescription and used for hair growth as described in this invention. Insulin is a hormone secreted by beta cells in the islets of Langerhans in the pancreas. It activates and participates in all the metabolic pathways in the normal and disease afflicted cells; however, it can lead to increased DNA, RNA and protein synthesis which result in increased growth by mitosis (Osborne C K, et al. Hormone responsive human breast cancer in long-term tissue culture: effect of insulin. Proc Natl Acad Sci USA 1976; 73: 4536-4540); enhances the permeability of cell membranes to many therapeutic agents (hair growth therapeutic agents) besides glucose, and electrolytes.

Insulin helps and facilitates to move the therapeutic, pharmaceutical, biochemical and biological agents molecules from extra cellular fluid (ECF) to intracellular fluid (ICE) meaning from outside the cells to inside the cells. It is anabolic trophic factor needed for the growth, multiplication of all cells in the body including hair growth stem cells in the hair follicle and multiplying matrix hair bulb cells of the scalp hair. Increased cellular metabolic activity induced by insulin also enhances the uptake and augment the action of all therapeutic, pharmaceutical, biochemical and biological agents by the cells and inside the cell including the cells responsible for scalp hair growth.

Insulin enhances the concentration and therapeutic effectiveness of therapeutic, pharmaceutical, biochemical and biological agents which has disease curing besides hair growing effect. Once inside the cells; the insulin enhances, and expands the effectiveness of any and all therapeutic agents including that agent proven to promote the hair growth such as minoxidil, finasteride; Latisse and other hair growth promoting prostaglandin preparations.

Our studies show that the Insulin itself enhances the hair growth on its own right. This is further supported by the observation of hair loss in diabetics and hair growth with hyperinsulinism. Our body has certain metabolic cycles, and one of these is the hair growth cycle as explained above. An individual hair will grow for several years and then enter a resting phase before being shed. In a healthy non-diabetic person, this fallen hair regrows quickly. However, diabetes affects the normal metabolic cycles of our body, including the hair growth cycle.

If this cycle is disrupted, hair that is shed normally may not regrow right away, or it may not regrow at all. Diabetes can also affect hair growth in these ways: This disease weakens immune system, increasing the susceptibility to infections, including scalp infections, which can cause hair loss. Diabetes also adversely affects the circulatory system (ASVD), causing problems all over your body, including hair follicles of the scalp. Hair follicles that don't get enough nutrients because of circulatory problems can't produce new hairs, and the follicles may even die from lack of nutrition. Local application of insulin to the hair growth desired area enhances the hair growth as described in this invention. Interestingly, the high levels insulin the blood in some diseases conditions and diabetics can result in hypertrichosis.

In our decade of studies and medical practice; we found, there is not a single disease which cannot be treated using Insulin to enhance the therapeutic effectiveness of the pharmaceutical, biochemical and biological agents' compounds including hair loss and augmentation of hair growth. In an ingenious vitro studies, it has been conclusively and methodically demonstrated that the Insulin activates and modifies metabolic pathways in MCF-7 human breast cancer cells, and increase the cytotoxic effect of methotrexate up to 10,000 fold (Oliver Alabaster' et al. Metabolic Modification by Insulin Enhances Methotrexate Cytotoxicity in MCF-7 Human Breast Cancer Cells, Eur J Cancer Clinic; 1981, Vol 17, pp 1223-1228. Richard L. Schilsky and Frederick. S. Ordway. Insulin effects on methotrexate polyglutamate synthesis and enzyme binding in cultured human breast cancer cells. Cancer Chemotherapy Pharmacol (1985) 15: 272-277).

Research studies in human breast cancer and my own studies on every kind of cancer and infection in any part of the body have shown that the group treated with insulin plus low dose methotrexate and other anticancer agents (and or antibiotics for infection) responded better than the patient treated with insulin or chemotherapy alone (Eduardo Lasalvia-Prisco et al. Insulin-induced enhancement of antitumoral response to methotrexate in breast cancer patients. Cancer Chemotherapy Pharmacol (2004) 53: 220-224. Ayre S G, Perez Garcia y Bellon D, Perez Garcia D Jr (1990) Neoadjuvant low-dose chemotherapy with Insulin in breast carcinomas. Eur J Cancer 26:1262-1263; T. R. Shantha; Effect of Insulin on cancer and infection: presented at Cancun 2nd IPT international conference on 2004 and unpublished studies).

These observations supports the hypothesis that disease or healthy cell sensitivity to therapeutic, pharmaceutical, biochemical and biological agents such as those to be used to enhance the hair growth could be increased by using the method described in this invention using insulin, and IGF-I. Our study of injecting Insulin followed by anticancer chemotherapeutic agents directly into cancer masses on hundreds of advanced and localized cancers supports this finding. Using this method, the palpable tumors including enlarged lymph nodes literally melted away. Insulin enhanced the therapeutic, pharmaceutical, biochemical and biological agent's activity many hundreds of folds, making them more effective. The same effects are observed when used with other hair growth products. We also used plain insulin locally as a therapeutic agent in chronic wounds, periodontal disease, post surgical wound healing, many eye and ear diseases etc which will be reported later.

The hormone IGF-1 (insulin-like growth factor 1) is a growth hormone that is believed to work with androgens to initiate and maintain hair growth. Working with androgens, IGF-1 may induce hair growth factors (Itami S, Kurata S, Takayasu S. Androgen induction of follicular epithelial cell growth is mediated via insulin-like growth factor-I from dermal papilla cells. Biochem Biophys Res Commun 1995 Jul. 26; 212(3):988-94). Researchers also suspect that IGF-1 may be able to stimulate the growth hair follicle cells directly (Su H Y, Hickford J G, Bickerstaffe R, Palmer B R. Insulin-like growth factor 1 and hair growth. Dermatol Online J 999 November; 5(2):1), and may regulate hair growth and the hair growth cycle on its own (Hembree J R, Harmon C S, Nevins T D, Eckert R L. Regulation of human dermal papilla cell production of insulin-like growth factor binding protein-3 by retinoic acid, glucocorticoids, and insulin-like growth factor-1. J Cell Physiol 1996 jun; 167(3):556-619.).

Insulin-like growth factor I (IGF-I) accelerates, in a concentration-dependent manner, growth of hair and hair follicles. Insulin-like growth factor (IGF)-I and -II had no significant effect on hair follicle growth when maintained in the presence of 10 micrograms/ml insulin. However, in the absence of insulin, both IGF-I (0.01-100 ng/ml) and IGF-II (0.01-100 ng/ml) stimulated hair follicle growth in a dose-dependent manner. IGF-I was more potent than either insulin or IGF-II, stimulating maximum rates of hair follicle growth at 10 ng/ml, whereas IGF-II gave maximum stimulation at 100 ng/ml. The rates of hair follicle growth stimulated by 10 ng/ml IGF-I were identical to those stimulated by 10 micrograms/ml insulin. IGF-II (100 ng/ml), however, was unable to stimulate hair follicle growth to the same extent as insulin. Both IGF-I (10 ng/ml) and IGF-II (100 ng/ml) were more potent than insulin at preventing hair follicles from entering into a catagen-like state. Relaxin is a member of the insulin family of polypeptide hormones and exerts its best understood actions in the mammalian reproductive system and may have an effect on rapid hair growth when combined with insulin and other known therapeutic, pharmaceutical, biochemical and biological agents. So the role of IGF-1 (also known as Somatomedin-C) in hair health cannot be ignored and can be used to enhance the growth of scalp hair as described in this invention.

The actions of IGF-I are modulated by proteins produced in dermal papilla cells which bind IGF (insulin-like growth factor-binding proteins: IGFBPs) which acts as mitogen. The exact mechanism of modulation has not yet been resolved (Batch J A, Mercuri F A, Werther G A. Identification and localization of insulin-like growth factor-binding protein (IGFBP) messenger RNAs in human hair follicle dermal papilla. J Invest Dermatol 1996; 106: 471-5.).

However, it has been shown that IGFBP-3 (which is the most abundant IGFBP type in dermal papilla cells) forms a complex with free IGF-I to reduce the concentration of IGF-I available for stimulation of hair elongation and maintenance of the anagen phase (Hembree J R, Harmon C S, Nevins T D, et al. Regulation of human dermal papilla cell production of insulin-like growth factor binding protein-3 by retinoic acid, glucocorticoids, and insulin-like growth factor-1. J Cell Physiol 1996; 167: 556-61). Retinoids and glucocorticoids stimulate production of IGFBP-3 in dermal papilla cells reducing the hair growth.

Insulin stimulated hair follicle growth in a dose-dependent manner over the range of 0.01 to 100 micrograms/ml. Maximum rates of hair follicle growth were observed when follicles were maintained in medium containing 10 micrograms/ml insulin, which is supraphysiologic. Hair follicles maintained in the absence of insulin or at physiologic levels showed premature entry into a catagen-like state (Philpott M P, Sanders D A, Kealey T. Effects of insulin and insulin-like growth factors on cultured human hair follicles: IGF-I at physiologic. J Invest Dermatol 1994; 102: 857-61). Insulin itself has the same effect as IGF-I. It has been observed that body hair in patients with hyperinsulinism has a male distribution pattern (Fossati P, Fontaine P. Endocrine and metabolic consequences of massive obesity. Rev Prat 1993; 43: 1935-9. Hrnciar J, Hrnciarova M, jakubikova K, et al. Insulin resistance and arterial hypertension. Hyperinsulinism as a basic etiopathogenic factor in essential arterial hypertension and associated phenomena. Vnitr Lek 1992; 38; 868-78). Effect of IGF-1 and their receptors are described in many articles as quoted here (Itami S, Kurata S, and Takayasu S. Androgen induction of follicular epithelial cell growth is mediated via insulin-like growth factor-1 from dermal papilla cells. Biochem Biophys Res Commun 212: 988-994, 1995). Itami S, Kurata S, and Takayasu S. Androgen induction of follicular epithelial cell growth is mediated via insulin-like growth factor-1 from dermal papilla cells. Biochem Biophys Res Commun 212: 988-994, 1995. Kamiya T, Shirai A, Kawashima S, Sato S, and Tamaoki T. Hair follicle elongation in organ culture of skin from newborn and adult mice. J Dermatol Sci 17: 54-60, 1998. Little J C, Westgate G E, Evans A, and Granger S P. Cytokine gene expression in intact anagen rat hair follicles. J Invest Dermatol 103: 715-720, 1994. Ruckert R, Hofmann U, Van Der Veen C, Bulfone-Paus S, and Paus R. MHC class I expression in skin: developmentally controlled and strikingly restricted intraepithelial expression during hair follicle morphogenesis and cycling, and response to cytokine treatment in vivo. J Invest Dermatol 111: 25-30, 1998. Stones A J, Granger S P, and Jenkins G. Localization of cytokines and their receptors in human hair follicles using immunogold histochemistry (Abstract). J Invest Dermatol 102: 627, 1994. Hodak E, Gottlieb A B, Anzilotti M, and Krueger J G. The insulin-like growth factor 1 receptor is expressed by epithelial cells with proliferative potential in human epidermis and skin appendages: correlation of increased expression with epidermal hyperplasia. J Invest Dermatol 106: 564-570, 1996)

Hyperinsulinism as a basic etiopathogenic factor in essential arterial hypertension, ASVD, abnormal hair growth on the body, and associated phenomena. Insulin has profound effect on cell mitosis and metabolism as described above. High levels of insulin in these patients stimulate the stem cells, and germinal matrix cells of the hair follicle contributing to this condition. How effective is the insulin in hair growth has been demonstrated that the hair follicle is an aerobic glycolytic, glutaminolytic tissue needing insulin for its health and growth. (Philpott M P, Sanders D, Westgate G E, Kealey T. Human hair growth in vitro: a model for the study of hair follicle biology. J Dermatol Sci. 1994 July; 7 Suppl:S55-72). Philpott et all studies showed; that in the absence of insulin hair follicles show premature entry into a catagen-like state which can be prevented by IGR-I. These studies support our invention that Insulin and IGF-1 can be used topically to prevent hair loss and accelerated hair growth.

Insulin will enhance the glucose uptake to produce energy in the form of ATP, which enhances all the metabolic activity of the entire stem and germinal cells the hair follicle leading to their mitosis and accelerated hair growth. Addition of insulin is known to induce mitosis in tissue cultures.

In an important experiment, Zheng et al showed the role of insulin like growth factor-I which have insulin like effect; induced the inner ear epithelial cell culture growth (Zheng, J. L., Helbig, C. & Gao, W-Q. Induction of cell proliferation by fibroblast and insulin-like growth factors in pure rat inner ear epithelial cell cultures. J. Neurosci. 17:216-226 (1997). There is a clear indication that insulin and IGF-I not only play a role in potentiation of pharmacological therapeutic effect of pharmaceutical, biochemical and biological agents, they can independently stimulate cells (inner ear hair cells) growth (T. R. Shantha, Unknown Health Risks of Inhaled Insulin. Life Extension, September 2007 pages 74-79, Post publication comments in September 2008 issue of Life Extension, Pages 24. T. R. Shantha and Jessica G. Inhalation Insulin, Oral and Nasal Insulin Sprays for Diabetics: Panacea or Evolving Future Health Disaster. Part I: Townsend Letter journal: Issue #305, December 2008 pages: 94-98; Part II: Townsend Letter, January, 2009, Issue #306, pages—106-110). These data suggest that in vitro IGF-I and insulin may be an important physiologic regulator of hair growth and possibly the hair growth cycle that can be used locally as described in this invention.

On the other hand; growth hormone (somatotrophin) alone has no direct influence on hair follicle and hair growth. It needs insulin to have its trophic effect exerted on the cells (Philpott M P, Sanders D A, Kealey T. Effects of insulin and insulin-like growth factors on cultured human hair follicles: IGF-I at physiologic. J Invest Dermatol 1994; 102: 857-61). Graying or whitening of hair is due to the collection of minute air bubbles in the cortex and medulla of the hair shaft and due to loss of pigment (melanin) formation by melanocytes cells in the germinal matrix.

It is a known physiological phenomenon that the insulin does bind to the receptor sites of the IGF-I and insulin receptors and exert multiple profound physiological effects (induce stem cells, and germinal cells growth of the hair follicle besides enhancing the glucose transport to produce ATP) such as enhanced metabolism, enhances mitosis, enhances the therapeutic effect of other pharmacological agents as reported (Shantha; T R: Unknown Health Risks of Inhaled Insulin LIFE EXTENSION SEPTEMBER 2007, Page 78-82) on the cell to which it binds has been reported in above publications. This physiological effect of insulin is used to turn the non dividing resting cancer cells to divide and make them sensitive to radiation therapy as well as anti metabolic chemotherapy agents and antibiotics. The same principle is used in the treatment of hair loss as described below in our invention.

Insulin, potassium and glucose are routinely administered to treat low potassium levels in the cells even to this day. Insulin and glucose facilitates the entry of potassium inside the cell—a life saving measure. We have used insulin as potentiator of uptake and enhancer of therapeutic action of diverse therapeutic agents to cure and/or curtail curable acute, chronic and incurable diseases such as cancers, Lyme disease, scleroderma, antibiotic resistant staphylococcus infection, MRSA infection, chronic wounds, neurological diseases, inner and middle ear affliction, autoimmune diseases and many other diseases with good results.

We have used insulin for more than a decade to enhance the effectiveness of locally injected therapeutic agents specially cancers with chemotherapeutic agents with remarkable results. Our research data also supports that the insulin spray on indolent ulcers anywhere in the body, including the oral (gums) and nasal cavity. It stimulated the fibroblast, endothelial cell and skin cell growth resulting accelerated wound healing. Application of insulin soaked cotton swabs (1-3-5 units in normal saline) after teeth extraction induces rapid healing with reduced pain. Our studies show that the application of insulin and antibiotics such as doxycycline locally on the gums eliminated gum diseases (periodontitis) which is under study.

During chemotherapy, spraying the head of the patients with insulin spray on the scalp with 3-5 units of insulin diluted normal saline or ringers lactate during chemotherapy, reduced the hair loss dramatically. It is this experience that leads us to this invention. After spray of the insulin, and drying of the spray on the scalp, scalp was tightly bound to reduce the blood circulation; hence the amount of chemotherapy bound blood reaching the hair follicles of the scalp. We also experimented with application of cold pack to the scalp 15 minutes after insulin spray, which caused the constriction of BV and reduced the amount of chemotherapy agent reaching the scalp hair which causes havoc on the hair root cells. These three simple methods reduced the profound loss of scalp hair to chemotherapy toxic effects in cancer patients during chemotherapy treatment. Insulin applied directly to the scalp had preventive effect on hair loss due to chemotherapy.

Insulin is a metabolic activity enhancer of all cells and therapeutic agents. Hence it can play an important role in treatment of hair loss (Dr. T. R. Shantha; 1. discovery of insulin and IPT: amazing history, 2. high dose methotrexate therapy using Insulin; 3 local injections of tumors with insulin and cytotoxic drugs; 4. two and three cycle insulin Potentiation therapy: Presented at 2^{nd} international conference on Insulin Potentiation Therapy held at Cancun, Mexico, Jun. 28-Jul. 1, 2004). A synergy between certain membrane and metabolic effects of insulin on cell molecular biology increases therapeutic efficacy, and it does so with reduced doses of the drugs, enhancing their uptake with augmented greater than before therapeutic efficacy and increasing safety as reported Oliver Alabaster’ et al.

A variety of facilitators (carriers, adjuvant agents, absorption enhancers and facilitators, assist to get entry into the cell, potentiators of therapeutic activity once in contact with the afflicted structures within the cells) and methods have been used to enhance the absorption and/or to potentiate the effect of therapeutic drugs administered to the patients for treatment of diseases. U.S. Pat. No. 2,145,869 disclose a method for the treatment of syphilis in general and neurosyphilis in particular. U.S. Pat. No. 4,277,465 discloses an insulin adjuvant to potentiate for treatment of diabetes. U.S. Pat. No. 4,196,196 discloses a composition of insulin, glucose and magnesium dipotassium ethylene diamine tetra acetic acid (EDTA) to enhance tissue perfusion and to facilitate a divalent/monovalent cation gradient. U.S. Pat No 4,971,951 discloses Insulin Potentiation Therapy (IPT) for the treatment of virally related diseases such as cancers and AIDS using insulin to deliver the drugs inside the cell with less or non toxic low doses of therapeutic, pharmaceutical, biochemical and biological agents. These prior art patents do not disclose and/or teach the use of insulin for hair growth as described in the present invention; for direct delivery of therapeutic, biological and diagnostic agents and to treat hair loss.

Insulin when applied to the bald scalp surface, enters the superficial layer of the skin, it enters the deeper layers of skin especially thorough the side of the hair shaft, follicle, sweat and sebaceous gland openings into the hair bulb and papilla where it exerts its hormonal effect on germinal multiplying cells and stem cells responsible of hair growth and their health (see FIGS. 1, 2). That is why, it is very important to keep the therapeutic intervention area completely dry so that the therapeutic, pharmaceutical, biochemical and biological agents especially insulin and IGF-1 used to stimulate and accelerate the hair growth are rapidly absorbed to the hair root where they exert their maximum effect on hair growth.

Insulin and IGF-I uptake into hair follicle can be enhanced combined with uptake facilitators such electroporation, iontophoresis, sonophoresis, vibroacoustic, vibration and other physical (heat, magnetic force, radio frequency, microwave, laser lights etc) methods with other appropriate therapeutic, biological and pharmacological hair growth agents combined with insulin. These methods can be used as prophylaxis, to diagnose and to treat scalp hair loss.

It is known that the pharmaceutically acceptable oxidizing agent facilitates the delivery of the bioactive agent through the skin and mucous membranes. In general, the oxidizing agent can react with molecules present in the skin that would adversely react with the bioactive agent. For example, reduced glutathione present in the skin can inactivate bioactive agents such as insulin by breaking chemical molecular bonds. Not wishing to be bound by theory, when delivering insulin transdermally, reduced glutathione can inactivate insulin and its can be counteracted by using oxidizing agents such as iodine, DMSO, hydrogen peroxide etc as described below.

Specifically, insulin has numerous disulfide bonds which are crucial for its protein conformation, biological activity, and subsequent therapeutic effects. Reduced glutathione will inactivate insulin by reducing or breaking insulin's disulfide bonds. Once these disulfide bonds are broken; insulin becomes inactive due to lost protein conformation and biological activity. Thus, the administration of the oxidant or oxidizing agents by spray, ointment, cream, or solution application or patch using the devices (as described by Shantha et al in U.S. PATENT APPLICATION PUB. NO.: 2009/0347776 A1) herein prevents the inactivation of the bioactive agent such as insulin when applied to the skin and eyelid edges. Specifically, applying an oxidant or a pharmaceutically oxidizing agent transdermally will lower or prevent the effects reduced proteins and reduced biological molecules have on the bioactive agents.

In this manner, the inactivation of bioactive agents via reduction or cleavage of crucial molecular bonds will be avoided. The selection and amount of the pharmaceutically acceptable oxidizing agent can vary depending upon the bioactive agent that is to be administered. In one aspect, the oxidizing agent includes, but is not limited to, iodine, povidine-iodine, and any source of iodine or combinations of oxidants, silver protein, active oxygen, potassium permanganate, hydrogen peroxide, sulfonamides, dimethyl sulfoxide (DMSO) or any combination thereof. These oxidizing agents may also act as absorption agents which help facilitate delivery of a therapeutic agent onto and into a skin.

In one aspect, the oxidant is at least greater than 1% weight per volume, weight per weight, or mole percent. In another aspect, the mucosal membrane permeability enhancer may be at least greater than 1.5%, 2.0%, 20 2.5%, 3.0%, 3.570, 4.0%, or 4.5% weight per volume, weight per weight, or mole percent. In this aspect, the oxidant may range from 2% to lo %, 2% to 9.5%, 3% to 8%, 3% to 7%, or 4% to 6% weight per volume, weight per weight, or by mole percent. Additional components can be present in the device to facilitate the delivery of the bioactive agent transdermally to the subject.

In one aspect, transdermal penetration enhances can be used to further expedite the entry of the bioactive agent into the hair follicle including hair papilla which is responsible for hair growth. Penetration enhancers not only penetrate a membrane efficiently, but these enhancers also enable other bioactive agents to cross a particular membrane more efficiently. Penetration enhancers produce their effect by various modalities such as disrupting the cellular layers of the alopecia skin surface interacting with intracellular proteins and lipids, or improving partitioning of bioactive agents as they come into contact with the hair follicle and its germinal multiplying cells.

With these enhancers, macromolecules up to 10 kDa are able to pass through the epidermal layers of skin to dermal layer reaching the hair papilla, follicle and bulb. These enhancers should be non-toxic, pharmacologically inert, non-allergic substances. In general these enhancers may include anionic surfactants, ureas, fatty acids, fatty alcohols, terpenes, cationic surfactants, nonionic surfactants, zwitterionic surfactants, polyols, amides, lactam, acetone, alcohols, and sugars. In one aspect, the 10 penetration enhancer includes dialkyl sulfoxides such as dimethyl sulfoxide (DMSO), decyl methyl sulfoxide, dodecyl dimethyl phosphine oxide, octyl methyl sulfoxide, nonyl methyl sulfoxide, undecyl methyl sulfoxide, sodium dodecyl sulfate and phenyl piperazine, or any combination thereof. In another aspect, the penentration enhancer may include lauryl alcohol, diisopropyl sebacate, oleyl alcohol, diethyl sebacate, dioctyl sebacate, dioctyl azelate, hexyl laurate, ethyl caprate, butyl stearate, dibutyl sebacate, dioctyl adipate, propylene glycol dipelargonate, ethyl laurate, butyl laurate, ethyl myristate, butyl myristate, isopropyl palmitate, isopropyl isostearate, 2-ethylhexyl pelargonate, butyl benzoate, benzyl benzoate, benzyl salicylate, dibutyl phthalate, or any combination thereof. In one aspect, the skin permeability enhancer is at least greater than 1% weight per volume, weight per weight, or mole percent. In another aspect, the mucosal membrane permeability enhancer may be at least greater than 1.5%, 2.O %, 2.S %, 3.0%, 3.5%, 4.0%, 4.5% up to 50% weight per volume, weight per weight, or mole percent. In one aspect, the mucosal membrane permeability enhancer is dimethyl sulfoxide. In this aspect, the amount of dimethyl sulfoxide may range from 2% to 10%, 2% to 9.5%, 3% to 8%, 3% to 7%, or 4% to 6% weight per volume, weight per weight, by mole percent, or any effective therapeutic amount.

In other aspects, these additional components may include antiseptics, antibiotics, anti-vitals, anti-fungals, anti-inflammatories, anti-parasitic, anti-dolorosa, antihistamines, steroids, vasodilators and/or vasoconstrictors can be applied topically to reduce inflammation, irritation, or reduce rapid absorption through the alopecia skin to the root of the hair which need to be activated to stimulate the hair growth and restore the hair loss. Such vasoconstrictors may include phenylephrine, ephedrine sulfate, epinephrine, naphazoline, neosynephrine, vasoxyl, oxyrnetazoline, or any 5 combination thereof. Such anti-inflammatories may include non-steroidal anti-inflammatory drugs (NSAIDs). NSAIDs alleviate pain and inflammation by counteracting cyclwxygenase and preventing the synthesis of prostaglandins.

In one aspect. NSAIDs include celecoxib, meloxicam, nabumetone, piroxicam, napmxen, oxaprozin, rofecoxib, sulindac, ketoprofen, valdewxid, anti-tumor necrosis factors, 10 anti-cpokines, anti-inflammatory pain causing bradykinins or any combination, thereof. Such antiseptics, anti-vitals, anti-fungals, and antibiotics, may include ethanol, propanol, isopropanol, or any combination thereof; a quaternary ammonium compounds including, but not limited to, benzalkonium chloride, cetyltrimethylammonium bromide, cetylpyridinium chloride, benzethonium chloride, or any combination thereof: boric acid; chlorhexidine gluconate, hydrogen peroxide, iodine, mercurochrome, ocetnidine dihydrochloride, sodium chloride, sodium hypochlorite, silver nitrate, colloidal silver, mupirocin, erthromycin, clindamycin, gentamicin, polymyxin, bacitracin, silver, sulfadiazine, or any combination thereof.

Alopecia is also contributed by the hair follicle, scalp, eye brows, and eye lash infection. It is intent of this invention to use of the insulin along with above described anti-inflammatory antibacterial agents that can eliminate one of the causes of hair loss and restore normal scalp hair growth.

In accordance with one aspect of the invention, the compound used to apply locally to the bald scalp site are mixed with a dermatologically well suited vehicle or carrier. The compositions of this invention may comprise aqueous solutions such as e.g., physiological saline, oil, gels, patches, solutions or ointments. The vehicle which carry these biologically active therapeutic agents such as insulin and IGF-I may contain dermatologically compatible preservatives such as e.g., benzalkonium chloride, surfactants like e.g., polysorbate 80, liposomes or polymers, for example, methyl cellulose, polyvinyl alcohol, polyvinyl pyrrolidone and hyaluronic acid etc. We have mixed 3-5 units of insulin in 5 ml of normal sline or pure water then used in the atomizer sprayer to spray on the bald area of the head.

There are various forms of insulin used to treat diabetes. They are grouped under rapid, short, intermediate, and long acting insulin as shown below in Table 1. It is also dispensed as premixed form containing rapid to long acting insulin. We use rapid and short acting insulin as noted in the following table to enhance the scalp hair growth.

TABLE 1
Types of Insulin we use in our invention and their
trade names TABLE 1

| | | | |
|---|---|---|---|
| Type of Insulin | | | |
| & Brand | Onset of | Peak | Duration |
| or trade Names | action | effect | of action |

Rapid-Acting or ultra short acting Insulin

| | | | | | |
|---|---|---|---|---|---|
| Humalog or lispro | 15-30 min. | 30-90 | min | 3-5 | hours |
| Novolog or aspart | 10-20 min. | 40-50 | min. | 3-5 | hours |
| Apidra or | 20-30 min. | 30-90 | min. | 1-2½ | hours |
| glulisine | | | | | |

Short-Acting Insulin

| | | | | | |
|---|---|---|---|---|---|
| Regular (R) | 30-60 min. | 2-5 | hours | 5-8 | hours |
| humulin or | | | | | |
| novolin | | | | | |

We found the use of rapid acting insulin marketed as Humalog or Novolog, regular humulin or novolin are best suited for treatment of hair growth. Our studies also found that local application of rapid action or other insulin formulations on the balding scalp did not change the blood sugar levels indicating it is safe to apply 3-5 IU insulin to the balding site at intervals of one hour.

Scalp Hair Growth Stimulant and Examples of how to Use Our Invention

The therapeutic agents described in this invention are applied at the emergence of vellus hair at the balding area.

Wash scalp with mild shampoo. Avoid using hair conditioners which may coat the bald skin resulting in prevention of the effective absorption of insulin. This is followed by application of oxidant. In our invention, we can use any one of the above described antioxidants, but in this instance we are discussing povidine iodine. We have also used insulin application without the use of oxidants which is as effective on the balding skin, only required application more often. Allow it to dry, and follow it by application of insulin by a spray or swab repeatedly 2-3 times as it dries.

This can be followed by application of hair growth therapeutic agents as described below which is already available FDA approved and/or those about to be approved or not approved. This process can be repeated every 12 or 24 hours for 3-7 days a week till the desirable results are obtained which may take up to 12 weeks or longer. Once the objective is achieved, use the method 2 to 3 times weekly before going to bed to keep the hair growth stimulation.

The hair growth stimulant of the present invention is obtained which are already in the market under prescription or under experiment.

Before starting the hair enhancing therapy, one needs to find out the underlying cause for the balding. Once the underling etiology is found, treat the disease if etiology is found and then use the hair growth product as described in this invention.

Preparations Before Application of Hair Growth Products of this Invention

The insulin preparations should be applied preferably at night time before going to bed or after morning shower. First the person must Wash the scalp with mild shampoo and dry it. Then apply the solution with cotton soaked in the solution or by the help of an atomizer spray. The bottles are containing therapeutic agents are supplied with multiple sterile applicators and instructions.

The side effect after application can be itching and redness which will go away with continued use. Use of simple insulin to enhance the hair growth did not result in any appreciable darkening of the skin as noticed with prostaglandin preparations on the lids. There was no change in blood glucose levels after application of the insulin to the scalp. If one develops hypoglycemic symptoms due to any number of reasons, drink a sugar drink, eat a candy, or fruit cocktail. We never have to use sugar to correct hypoglycemia because none of our cases developed hypoglycemia.

The Following Steps Will Help to Use the Products of this Invention

Step 1: Wash the entire scalp, remove oily applications on the balding area with mild shampoo and dry the site of therapeutic intervention.

Step 2: Apply Oxidant to the affected area with a Q tip or triangular applicator or cotton swab or use an atomizer spray with the medications of this invention. In this aspect, the oxidant may range from 2% to 10%, 2% to 9.5%, 3% to 8%, 3% to 7%, or 4% to 6% weight per volume, weight per weight, or by mole percent. In this aspect, the oxidant may range from 2% to 10%, 2% to 9.5%, 3% to 8%, 3% to 7%, or 4% to 6% weight per volume, weight per weight, or by mole percent. It is our observation, that using insulin on the may not need an oxidant for insulin and other hair growth products to be effective on the hair follicle and hair bulb. This is because, the hair roots are superficially place and there is easy access to the insulin and other therapeutic agents to get access to act on the hair follicle to grow. Hence the step 2 is optional and may not be part of the example we describe below.

Step 3: Apply insulin by sterile Q tip or triangular applicator or cotton swab or atomizer spray after the Oxidant (and/or permeability enhancer) are applied. Insulin can be in the form of watery dilute solution, gel or semi liquid or ointment can be applied without any of the absorption enhancers or oxidants. We prefer to use watery liquid spray or coating with cotton swab.

Step 4: Allow the insulin to get absorbed and act on hair follicles for 15 minutes and then apply the selected hair growth factor (hypertrichotic effect) on the bald surface and leave it to be absorbed. The application of this therapeutic, pharmaceutical, biochemical and biological agent with insulin can be repeated after 4-12 hours if rapid hair growth is desired.

Step 5: Optional step: Absorption enhancers, vasodilators, anti-inflammatory therapeutic agents can be applied if there is an underlying cause affecting the hair loss or noticeable slow growth. This step can be before, along with or after the application of therapeutic, pharmaceutical, biochemical and biological agents which promotes the hair growth.

Though we give an example of 5 step process of using our invention, the step can be summarized as follows:

Step 1: Wash the scalp and remove oily applications on the balding area and dry the site of therapeutic intervention.

Step 2: Apply selected oxidant to the balding scalp. This step is optional and we have avoided it in many cases.

Step 3: Apply insulin by using sterile Q tip or triangular applicator or cotton swab or using aerosol atomizer spray. Repeatedly apply it as it dries out so that the cells of the hair follicle absorb it completely in high concentrations.

Step 4: Apply selected hair growth therapeutic agent besides insulin if desired or advised.

Step 5: Optional step: Absorption enhancers, vasodilators, anti-inflammatory therapeutic agents can be applied if there is an underlying cause affecting the hair loss.

These steps can be repeated 2-3 times a day if need be and best applied at bed time.

### Example 1

Application of insulin alone as hair growth factor without other therapeutic agents.

Step 1: Wash the entire scalp, remove oily substances on the balding area with mild shampoo and dry the site of therapeutic intervention thoroughly. It is better to shampoo, dry, and wait for 30 minutes for the water soaked superficial layers of the skin to be dried out completely to enhance the uptake of locally applied hair growth therapeutic agents.

Step 2: Apply oxidant to the balding site with a sprayer or cotton swab. It is an optional step.

Step 3: Apply insulin by Q tip or triangular applicator or cotton swab or by use of an atomizer sprayer. Repeatedly apply insulin as it dries out so that the cells of the hair follicle absorb it and get stem cells and matrix germinal cells stimulated to multiply and grow to enhance the hair growth.

Step 4: No other hair growth enhancer is used. Only insulin is used as hair growth accelerant.

Step 5: Optional step: Absorption enhancers, vasodilators, anti-inflammatory therapeutic agents can be applied if there is an underlying cause affecting the hair loss. This step can be pursued before, during or after the therapeutic agents' application.

### Example 2

Application of insulin with other therapeutic agents.

Step 1: Wash the entire scalp, remove oily substances on the balding area with mild shampoo and dry the site of therapeutic intervention thoroughly. It is better to shampoo, dry, and wait for 30 minutes for the water soaked superficial layers of the skin to be dried out completely to enhance the uptake of locally applied hair growth therapeutic agents.

Step 2: Apply oxidant to the balding site with a sprayer or cotton swab. It is an optional step.

Step 3: Apply insulin by Q tip or triangular applicator or cotton swab or by use of a atomizer sprayer. Repeatedly apply insulin as it dries out so that the cells of the hair follicle absorb it and get stem cells and matrix germinal cells stimulated to multiply and grow to enhance the hair growth.

Step 4: Apply selected hair growth therapeutic agent. In this present example we choose to use Minoxidil liquid, gel or cream. This therapeutic agent is already available in foam form for application to the balding scalp without prescription. Pretreatment of insulin before application will enhance the hair growth much rapidly in a short duration. They hair that are growing from using our method are robust, resulting in dark and rapid growth.

Step 5: Optional step: Absorption enhancers, vasodilators, anti-inflammatory therapeutic agents can be applied if there is an underlying cause affecting the hair loss. This step can be pursued before, during or after the therapeutic agents' application.

### Example 3

Application of insulin with other therapeutic agents.

Step 1: Wash the entire scalp, remove oily substances on the balding area with mild shampoo and dry the site of therapeutic intervention thoroughly. It is better to shampoo, dry, and wait for 30 minutes for the water soaked superficial layers of the skin to be dried out completely to enhance the uptake of locally applied hair growth therapeutic agents.

Step 2: Apply oxidant to the balding site with a sprayer or cotton swab. It is an optional step.

Step 4: Apply finasteride (Propecia) as hair growth enhancer is used.

Step 3: Apply insulin by Q tip or triangular applicator or cotton swab or by use of a atomizer sprayer. Repeatedly apply insulin as it dries out so that the cells of the hair follicle absorb it and get stem cells and matrix germinal cells stimulated to multiply and grow to enhance the hair growth.

Step 5: Optional step: Absorption enhancers, vasodilators, anti-inflammatory therapeutic agents can be applied if there is an underlying cause affecting the hair loss. This step can be pursued before, during or after the therapeutic agents' application.

### Example 4

Application of insulin hair growth factor with other therapeutic agents.

Step 1: Wash the entire scalp, remove oily substances on the balding area with mild shampoo and dry the site of therapeutic intervention thoroughly. It is better to shampoo, dry, and wait for 30 minutes for the water soaked superficial layers of the skin to be dried out completely to enhance the uptake of locally applied hair growth therapeutic agents.

Step 2: Apply oxidant to the balding site with a sprayer or cotton swab. It is an optional step.

Step 3: Apply insulin by Q tip or triangular applicator or cotton swab or by use of an atomizer sprayer. Repeatedly apply insulin as it dries out so that the cells of the hair follicle absorb it and get stem cells and matrix germinal cells stimulated to multiply and grow to enhance the hair growth.

Step 4: Apply selected hair growth therapeutic agent. In this present example we choose to use P-1075, (cromakalim), diazoxide or nicorandil instead of Minoxidil liquid, gel or cream. All these 3 therapeutic agents exert their hypertrichotic effect as potassium channel agonist in the vascular smooth muscles causing vasodilatation and enhanced blood supply and oxygen to the hair papilla and follicle. Any and all available Potassium channel agonists can be used instead these three known available therapeutic agents.

Step 5: Optional step: Absorption enhancers, vasodilators, anti-inflammatory therapeutic agents can be applied if there is an underlying cause affecting the hair loss. This step can be used before, during or after the therapeutic agents' application.

### Example 5

Local application of Insulin and cyclosporine, and hexachlorobenzene; Anabolic steroids Danazol.

Step 1: Wash the entire scalp, remove oily substances on the balding area with mild shampoo and dry the site of therapeutic intervention thoroughly. It is better to shampoo, dry, and wait for 30 minutes for the water soaked superficial layers of the skin to be dried out completely to enhance the uptake of locally applied hair growth therapeutic agents.

Step 2: Apply oxidant to the balding site with a sprayer or cotton swab. It is an optional step.

Step 3: Apply insulin by Q tip or triangular applicator or soaked cotton swab or by use of a atomizer sprayer. Repeatedly apply insulin as it dries out so that the cells of the hair follicle absorb it and get stem cells and matrix germinal cells stimulated to multiply and grow to enhance the hair growth.

Step 4: Apply selected hair growth therapeutic agent. In this present example we choose Cyproterone Acetate (CPA). It is steroidal androgen blocker. Taking oral forms will have feminizing effect in males, hence only ointment, gel or foam, liquid form needs to be formulated and used. Cyclosporine, and hexachlorobenzene; Anabolic steroids; Danazol can also be applied for hair growth locally after insulin application.

Step 5: Optional step: Absorption enhancers, vasodilators, anti-inflammatory therapeutic agents can be applied if there is an underlying cause affecting the hair loss. This step can be pursued before, during or after the therapeutic agents' application.

### Example 6

Application of Insulin and aromatase inhibitors.

Step 1: Wash the entire scalp, remove oily substances on the balding area with mild shampoo and dry the site of therapeutic intervention thoroughly. It is better to shampoo, dry, and wait for 30 minutes for the water soaked superficial layers of the skin to be dried out completely to enhance the uptake of locally applied hair growth therapeutic agents.

Step 2: Apply oxidant to the balding site with a sprayer or cotton swab. It is an optional step.

Step 3: Apply insulin by Q tip or triangular applicator or soaked cotton swab or by use of a atomizer sprayer. Repeatedly apply insulin as it dries out so that the cells of the hair follicle absorb it and get stem cells and matrix germinal cells stimulated to multiply and grow to enhance the hair growth.

Step 4: Apply selected hair growth therapeutic agent. In this present example we choose to use aromatase inhibitors which work by inhibiting the action of the enzyme aromatase, which converts androgens into estrogens by a process called aromatization. As breast tissue is stimulated by estrogens, decreasing their production is a way of suppressing recurrence of the breast tumor tissue. There are numerous Aromatase inhibitors such as: Testolactone (Teslac), Anastrozole (Arimidex), Letrozole (Femara), Exemestane, (Aromasin), Vorozole (Rivizor), Formestane (Lentaron), Fadrozole (Afema), 4-androstene-3,6,17-trione (“6-OXO”, marketed as a nutritional supplement for athletes and weight lifters), 1,4,6-androstatrien-3,17-dione (ATD), 4-hydroxyandrostenedione, Aminoglutethimide, 4-OH androstenedione, 6-hydroximino-andmstenedione, 9,10-dimethyl 1,2-benzanthraccne, 1,4,6-androstatrien-3,17-dione, Atamestane, Exemstane, Fadrozolc, 1,2,4-triazole-3-alamine, Rogletimide and many are available in the market. We can select any one of the suitable therapeutic agent for local application after topical insulin application preparation of step 3. Natural Aromatase inhibitors: Quercetin, naringenin, resveratrol, apigenin, genistein, and oleuropein are all powerful flavonoids from whole foods that inhibit aromatase while at the same time offering a treasure chest of other health benefits can also be used for hair growth.

Step 5: Optional step: Absorption enhancers, vasodilators, anti-inflammatory therapeutic agents can be applied if there is an underlying cause affecting the hair loss. This step can be pursued before, during or after the therapeutic agents' application.

### Example 7

Application of insulin hair growth factor with other therapeutic agents.

Step 1: Wash the entire scalp, remove oily substances on the balding area with mild shampoo and dry the site of therapeutic intervention thoroughly. It is better to shampoo, dry, and wait for 30 minutes for the water soaked superficial layers of the skin to be dried out completely to enhance the uptake of locally applied hair growth therapeutic agents.

Step 2: Apply oxidant to the balding site with a sprayer or cotton swab. It is an optional step.

Step 3: Apply insulin by Q tip or triangular applicator or soaked cotton swab or by use of a atomizer sprayer. Repeatedly apply insulin as it dries out so that the cells of the hair follicle absorb it and get stem cells and matrix germinal cells stimulated to multiply and grow to enhance the hair growth.

Step 4: Apply selected hair growth therapeutic agent. It is another intent of this invention to add additional growth factors known to promote growth of hair; such factors include, without limitation, insulin, insulin-like growth factor (IGF-1), Relaxin, interleukin-4 (IL-4), transforming growth factor (TGF) (e.g., TGFa or TGFPI), basic fibroblast growth factor (bFGF), epidermal growth factor (EGF), platelet-derived growth factor (PDGF), prolactin, neurotrophin, Hepatocyte growth factor, Alkaline phosphatase, or biotin.

Step 5: Optional step: Absorption enhancers, vasodilators, anti-inflammatory therapeutic agents can be applied if there is an underlying cause affecting the hair loss. This step can be pursued before, during or after the therapeutic agents' application.

### Example 8

Application of insulin hair growth factor with other therapeutic agent such as Deferoxamine.

Step 1: Wash the entire scalp, remove oily substances on the balding area with mild shampoo and dry the site of therapeutic intervention thoroughly. It is better to shampoo, dry, and wait for 30 minutes for the water soaked superficial layers of the skin to be dried out completely to enhance the uptake of locally applied hair growth therapeutic agents.

Step 2: Apply oxidant to the balding site with a sprayer or cotton swab. It is an optional step.

Step 3: Apply insulin by Q tip or triangular applicator or soaked cotton swab or by use of a atomizer sprayer. Repeatedly apply insulin as it dries out so that the cells of the hair follicle absorb it and get stem cells and matrix germinal cells stimulated to multiply and grow to enhance the hair growth.

Step 4: Apply selected hair growth therapeutic agent such as Deferoxamine; also known as desferoxamine B, desferoxamine B, DFO-B, DFOA, DFB or desferal. It is a bacterial siderophore produced by the actinobacter Streptomyces pilosus. It has medical applications as a chelating agent used to remove excess iron (and aluminium) from the body (Miller, Marvin J. 1989. “Syntheses and therapeutic potential of hydroxamic acid based siderophores and analogs”. Chemical Reviews 89 (7): 1563-1579). The mesylate salt of DFO-B is commercially available that can be applied on scalp. The dilute solution can also be injected subcutaneously to bald are of the scalp in very small doses. Deferoxamine acts by binding free iron in the hair follicle location of papilla and remove to the bloodstream and enhancing its elimination in the urine. By removing excess iron, the agent reduces the damage done to various organs and tissues by iron mediated free radicals' damage and enhances angiogenesis'. Studies at University of Alabama have shown that has angiogenic effect, hence can increase the blood supply to the hair papilla to promote hair growth. (http://www.sciencedaily.com/releases/2008/01/12.080110085148. htm). A recent study also shows that it speeds healing of nerve damage (and minimizes the extent of recent nerve trauma) and bones and may modulate expression and release of inflammatory mediators by specific cell type (Lee H J, Lee J, Lee S K, Lee S K, Kim E C. Differential regulation of iron chelator-induced IL-8 synthesis via MAP kinase and NF-kappaB in immortalized and malignant oral keratinocytes. BMC Cancer. 2007 Sep. 13; 7:176. PMID: 17850672. Choi E Y, Kim E C, Oh H M, Kim S, Lee H J, Cho E Y, Yoon K H, Kim E A, Han W C, Choi S C, Hwang J Y, Park C, Oh B S, Kim Y, Kimm K C, Park K I, Chung H T, Jun C D. Iron chelator triggers inflammatory signals in human intestinal epithelial cells: involvement of p38 and extracellular signal-regulated kinase signaling pathways. J Immunol. 2004 Jun. 1; 172(11):7069-77. PMID: 15153529).

Step 5: Optional step: Absorption enhancers, vasodilators, anti-inflammatory therapeutic agents can be applied if there is an underlying cause affecting the hair loss. This step can be pursued before, during or after the therapeutic agents' application.

### Example 9

Application of insulin hair growth factor with and hair follicle transplant.

Step 1: Wash the entire scalp, remove oily substances on the balding area with mild shampoo and dry the site of therapeutic intervention thoroughly. It is better to shampoo, dry, and wait for 30 minutes for the water soaked superficial layers of the skin to be dried out completely to enhance the uptake of locally applied hair growth therapeutic agents.

Step 2: Apply oxidant to the balding site with a sprayer or cotton swab. It is an optional step.

Step 3: Apply insulin by Q tip or triangular applicator or soaked cotton swab or by use of a atomizer sprayer. Repeatedly apply insulin as it dries out so that the cells of the hair follicle absorb it and get stem cells and matrix germinal cells stimulated to multiply and grow to enhance the hair growth.

Step 4: In our experience, we do not need a second hypertrichosis therapeutic agent. Insulin is enough to facilitate rapid wound healing along with uptake and growth of hair follicle transplant or hair transplant plugs or other hair restoring surgical method such as transplantation of adipocytes which has the capacity to grow hair. This method results regeneration and taking of all transplanted hair follicle containing skin with vigorous healthy sprouting of hair follicles with rapid growth once the hair shaft emerge from the dermis and epidermis. Apply the Wash dressing on the wound if need be.

Step 5: Optional step: Absorption enhancers, vasodilators, anti-inflammatory therapeutic agents can be applied if there is an underlying cause affecting the hair loss. This step can be pursued before, during or after the therapeutic agents' application.

### Example 10

Application of insulin hair growth factor with other therapeutic agents such as prostaglandins and their derivatives.

Step 1: Wash the entire scalp, remove oily substances on the balding area with mild shampoo and dry the site of therapeutic intervention thoroughly. It is better to shampoo, dry, and wait for 30 minutes for the water soaked superficial layers of the skin to be dried out completely to enhance the uptake of locally applied hair growth therapeutic agents.

Step 2: Apply oxidant to the balding site with a sprayer or cotton swab. It is an optional step.

Step 3: Apply insulin by Q tip or triangular applicator or soaked cotton swab or by use of a atomizer sprayer. Repeatedly apply insulin as it dries out so that the cells of the hair follicle absorb it and get stem cells and matrix germinal cells stimulated to multiply and grow to enhance the hair growth.

Step 4: Apply selected hair growth therapeutic agent, which includes prostaglandin and its derivatives which are already in the market and FDA approved. Prostaglandins and derivatives thereof are useful in a method of enhancing hair growth. Application of the hair growth promoting prostaglandins accelerates the robust hair growth compared to when used without insulin.

Step 5: Optional step: Absorption enhancers, vasodilators, anti-inflammatory therapeutic agents can be applied if there is an underlying cause affecting the hair loss. This step can be pursued before, during or after the therapeutic agents' application.

### Example 11

Application of insulin hair growth factor with other therapeutic agents such as extracts of Cotinus coggygria.

Step 1: Wash the entire scalp, remove oily substances on the balding area with mild shampoo and dry the site of therapeutic intervention thoroughly. It is better to shampoo, dry, and wait for 30 minutes for the water soaked superficial layers of the skin to be dried out completely to enhance the uptake of locally applied hair growth therapeutic agents.

Step 2: Apply oxidant to the balding site with a sprayer or cotton swab. It is an optional step.

Step 3: Apply insulin by Q tip or triangular applicator or soaked cotton swab or by use of an atomizer sprayer. Repeatedly apply insulin as it dries out so that the cells of the hair follicle absorb it and get stem cells and matrix germinal cells stimulated to multiply and grow to enhance the hair growth.

Step 4: Apply extracts of Cotinus coggygria; a selected hair growth and hair quality improving agent in an amount effective to induce hair growth when applied topically to an area of the lid skin on which hair growth is desired as disclosed in U.S. PATENT APPLICATION PUB. NO.: US 200810145331 A1.

Step 5: Optional step: Absorption enhancers, vasodilators, anti-inflammatory therapeutic agents can be applied if there is an underlying cause affecting the hair loss. This step can be pursued before, during or after the therapeutic agents' application.

### Example 12

Application of insulin hair growth factor with other therapeutic agents.

Step 1: Wash the entire scalp, remove oily substances on the balding area with mild shampoo and dry the site of therapeutic intervention thoroughly. It is better to shampoo, dry, and wait for 30 minutes for the water soaked superficial layers of the skin to be dried out completely to enhance the uptake of locally applied hair growth therapeutic agents.

Step 2: Apply oxidant to the balding site with a sprayer or cotton swab. It is an optional step.

Step 3: Apply insulin by Q tip or triangular applicator or soaked cotton swab or by use of an atomizer sprayer. Repeatedly apply insulin as it dries out so that the cells of the hair follicle absorb it and get stem cells and matrix germinal cells stimulated to multiply and grow to enhance the hair growth.

Step 4: Use a device that targets a specific site on the scalp which helps limit and/or eliminate concerns about the cosmeceuticals active ingredient effecting unintended areas of the body. Insulin and IGF-I uptake into hair follicle can be enhanced combined with uptake facilitators such electroporation, iontophoresis, sonophoresis, vibroacoustic, vibration and other physical (heat, magnetic force, radio frequency, microwave, laser lights etc) methods with other appropriate therapeutic, biological and pharmacological hair growth agents combined with insulin. These methods can be used as prophylaxis, to diagnose and to treat scalp hair loss.

Step 5: Optional step: Absorption enhancers, vasodilators, anti-inflammatory therapeutic agents can be applied if there is an underlying cause affecting the hair loss. This step can be pursued before, during or after the therapeutic agents' application.

### Example 13

Application of insulin hair growth factor with other therapeutic agents.

Step 1: Wash the entire scalp, remove oily substances on the balding area with mild shampoo and dry the site of therapeutic intervention thoroughly. It is better to shampoo, dry, and wait for 30 minutes for the water soaked superficial layers of the skin to be dried out completely to enhance the uptake of locally applied hair growth therapeutic agents.

Step 2: Apply oxidant to the balding site with a sprayer or cotton swab. It is an optional step.

Step 3: Apply insulin by Q tip or triangular applicator or soaked cotton swab or by use of an atomizer sprayer. Repeatedly apply insulin as it dries out so that the cells of the hair follicle absorb it and get stem cells and matrix germinal cells stimulated to multiply and grow to enhance the hair growth.

Step 4: Using a photodynamic therapy (PDT) to stimulate an increase in hair count numbers and restore hair growth in areas of hair loss. A method according to the present invention comprises: application of insulin or IGF-I at the site of hair loss and then inject locally or systemically the photosensitive material and apply the light to the desired site of hair loss. The insulin will enhance the uptake of the photosensitive material to become more effective in PDT as follows: a) Administering an effective and/or sufficient amount of a photosensitizer to the target skin; the insulin priming will allow the photosensitizer to be distributed at the site of dermal papilla evenly; b) irradiating the target skin with energy comprising one or more wavelength capable of activating said photosensitizer for a time period sufficient to in the treated area. In one aspect of the present invention, there is a 2% or more increase in the number of terminal hairs within 3 months. If used, pretreatment with insulin will enhance the effectiveness of the PDT.

Step 5: Optional step: Absorption enhancers, vasodilators, anti-inflammatory therapeutic agents can be applied if there is an underlying cause affecting the hair loss. This step can be pursued before, during or after the therapeutic agents' application.

### Example 14

Application of insulin hair growth factor with other therapeutic agents such as local application of tocotrienols.

Step 1: Wash the entire scalp, remove oily substances on the balding area with mild shampoo and dry the site of therapeutic intervention thoroughly. It is better to shampoo, dry, and wait for 30 minutes for the water soaked superficial layers of the skin to be dried out completely to enhance the uptake of locally applied hair growth therapeutic agents.

Step 2: Apply oxidant to the balding site with a sprayer or cotton swab. It is an optional step.

Step 3: Apply insulin by Q tip or triangular applicator or soaked cotton swab or by use of an atomizer sprayer. Repeatedly apply insulin as it dries out so that the cells of the hair follicle absorb it and get stem cells and matrix germinal cells stimulated to multiply and grow to enhance the hair growth.

Step 4: Tocotrienol is a form of vitamin E. It is a potent anti-oxidant and has been used in combating many health problems. There was a report of the beneficial effects of vitamin E in hair care products (Shipp J Jesus; 1994. Hair care products. In: Chemistry and technology of the cosmetics and toiletries industry. Williams D F and Schmitt W H, ed.s), p 66. Blackie Academic & Professional: UK.). U.S. PATENT APPLICATION PUB. NO.: 200410009135 A1 discloses the local application of tocotrienols. We recommend the use of tocotrienols after insulin application so that this therapeutic agent can easily permeate the hair follicle and promote the hair growth. Another antioxidant we have used effectively is an extract of turmeric, called curcumin after application of insulin.

Step 5: Optional step: Absorption enhancers, vasodilators, anti-inflammatory therapeutic agents can be applied if there is an underlying cause affecting the hair loss. This step can be pursued before, during or after the therapeutic agents' application.

### Example 15

Application of insulin hair growth factor with other therapeutic agents such as adipose tissue implanted to the balding area.

Step 1: Wash the entire scalp, remove oily substances on the balding area with mild shampoo and dry the site of therapeutic intervention thoroughly. It is better to shampoo, dry, and wait for 30 minutes for the water soaked superficial layers of the skin to be dried out completely to enhance the uptake of locally applied hair growth therapeutic agents.

Step 2: Apply oxidant to the balding site with a sprayer or cotton swab. It is an optional step.

Step 3: Apply insulin by Q tip or triangular applicator or soaked cotton swab or by use of an atomizer sprayer. Repeatedly apply insulin as it dries out so that the cells of the hair follicle absorb it and get stem cells and matrix germinal cells stimulated to multiply and grow to enhance the hair growth.

Step 4: The roots of actively growing hairs are embedded in a layer of fat cells (adipocytes). The U.S. PATENT APPLICATION PUB. NO.: 200310147831 A1 inventors noted that, while balding areas of the scalp are generally depleted of fat tissue, the occipital area of the scalp (in which balding seldom occurs) contains a thick layer of fat tissue. They considered it likely that fat cells produce a growth factor that is essential for hair growth. They are attempting to use hair follicle with adipose tissue to be implanted to the balding area. Application of insulin post surgical; make the hair root take the scalp and emerge as terminal hair very rapidly.

Step 5: Optional step: Absorption enhancers, vasodilators, anti-inflammatory therapeutic agents can be applied if there is an underlying cause affecting the hair loss. This step can be pursued before, during or after the therapeutic agents' application.

### Example 16

Application of insulin hair growth factor with other therapeutic agents such as a 5a-reductase inhibitor.

Step 1: Wash the entire scalp, remove oily substances on the balding area with mild shampoo and dry the site of therapeutic intervention thoroughly. It is better to shampoo, dry, and wait for 30 minutes for the water soaked superficial layers of the skin to be dried out completely to enhance the uptake of locally applied hair growth therapeutic agents.

Step 2: Apply oxidant to the balding site with a sprayer or cotton swab. It is an optional step.

Step 3: Apply insulin by Q tip or triangular applicator or soaked cotton swab or by use of an atomizer sprayer. Repeatedly apply insulin as it dries out so that the cells of the hair follicle absorb it and get stem cells and matrix germinal cells stimulated to multiply and grow to enhance the hair growth.

Step 4: No other hair growth enhancer is used. Only insulin is used as hair growth accelerant.

Step 4: Apply a selected hair growth therapeutic agent such as a hair growth stimulant comprising p-menthane-3,8-diol and at least one substance from the group of a blood circulation promoter, a 5a-reductase inhibitor (Examples of the 5-a-reductase inhibitor P-glycyrrhetic acid, estradiol and estrone;), an antihistaminic (diphenhydramine hydrochloride), a cell activator (hair follicular function include pantothenyl alcohol, pantothenyl ethyl ether, Photosensitizer 301 and extract of ginseng); those of the antiphlogistic include glycynhetic acid and derivatives those of the antimicrobial include hinokitiol and isopropyl methyl phenol in combination. The hair growth stimulant is free from hormonal action, does not cause side effects and has excellent hair growth promoting effects.

Step 5: Optional step: Absorption enhancers, vasodilators, anti-inflammatory therapeutic agents can be applied if there is an underlying cause affecting the hair loss. This step can be pursued before, during or after the therapeutic agents' application.

### Example 17

Application of insulin hair growth factor with other therapeutic agents such as agent comprising a processed semi-mature soybean and/or a processed semi-mature soybean extracts.

Step 1: Wash the entire scalp, remove oily substances on the balding area with mild shampoo and dry the site of therapeutic intervention thoroughly. It is better to shampoo, dry, and wait for 30 minutes for the water soaked superficial layers of the skin to be dried out completely to enhance the uptake of locally applied hair growth therapeutic agents.

Step 2: Apply oxidant to the balding site with a sprayer or cotton swab. It is an optional step.

Step 3: Apply insulin by Q tip or triangular applicator or soaked cotton swab or by use of an atomizer sprayer. Repeatedly apply insulin as it dries out so that the cells of the hair follicle absorb it and get stem cells and matrix germinal cells stimulated to multiply and grow to enhance the hair growth.

Step 4: Apply a selected hair growth therapeutic agent characterized by using a processed semi-mature soybean and/or a processed semi-mature soybean extracts and at least one substance selected from the group consisting of a processed Polygoni Multiflori Radix, processed Polygoni Multiflori Radix extracts, a processed Cynanchum bungei Decne or processed Cynanchum bungei Decne extracts, preferably further comprising Longan seed and or Longan seed extracts as active ingredients. This agent for hair growth has no side effects when used externally or internally, it can notably improve hair growth within a short period of time; ranging from 6 to 12 weeks, can return hair to its normal hair color (for example from white to black) and can improve the gloss of hair. This hair tonic can be more effective using our invention of preparing the site of hair loss with insulin and then apply the products described in this invention.

Step 5: Optional step: Absorption enhancers, vasodilators, anti-inflammatory therapeutic agents can be applied if there is an underlying cause affecting the hair loss. This step can be pursued before, during or after the therapeutic agents' application.

### Example 18

Application of insulin hair growth factor with other therapeutic agents such as an ethanol or aqueous ethanol preparation of fatty acid, fatty acid ester, polyglycerin fatty acid esters and at least one of sorbitan fatty acid esters.

Step 1: Wash the entire scalp, remove oily substances on the balding area with mild shampoo and dry the site of therapeutic intervention thoroughly. It is better to shampoo, dry, and wait for 30 minutes for the water soaked superficial layers of the skin to be dried out completely to enhance the uptake of locally applied hair growth therapeutic agents.

Step 2: Apply oxidant to the balding site with a sprayer or cotton swab. It is an optional step.

Step 3: Apply insulin by Q tip or triangular applicator or soaked cotton swab or by use of a atomizer sprayer. Repeatedly apply insulin as it dries out so that the cells of the hair follicle absorb it and get stem cells and matrix germinal cells stimulated to multiply and grow to enhance the hair growth.

Step 4: Apply a selected hair growth therapeutic which is an ethanol or aqueous ethanol preparation comprising, as active ingredients for a hair growth promotor, (A) at least one compound selected from fatty acids having a chain length of an odd number of carbon atoms, the derivatives of the fatty acids, aliphatic alcohols having a chain length of an odd number of carbon atoms and the derivatives of the aliphatic alcohols and (B) at least one selected from 6-benzylaminopurine and/or the derivatives thereof represented by the following Formula (I), wherein it further comprises (C) at least one of polyglycerin fatty acid esters and at least one of sorbitan fatty acid esters: in Formula (I), R, and R, are defined. The hair growth promoter can have a good hair growth effect and can provide an excellent stabilization effect at low temperature and can provide good feeling having no stickiness. Use of it after insulin application can enhance the effectiveness of above therapeutic agents.

Step 5: Optional step: Absorption enhancers, vasodilators, anti-inflammatory therapeutic agents can be applied if there is an underlying cause affecting the hair loss. This step can be pursued before, during or after the therapeutic agents' application.

### Example 19

Application of insulin hair growth factor with other therapeutic agents such as a vascular endothelial growth factor (VEGF).

Step 1: Wash the entire scalp, remove oily substances on the balding area with mild shampoo and dry the site of therapeutic intervention thoroughly. It is better to shampoo, dry, and wait for 30 minutes for the water soaked superficial layers of the skin to be dried out completely to enhance the uptake of locally applied hair growth therapeutic agents.

Step 2: Apply oxidant to the balding site with a sprayer or cotton swab. It is an optional step.

Step 3: Apply insulin by Q tip or triangular applicator or soaked cotton swab or by use of an atomizer sprayer. Repeatedly apply insulin as it dries out so that the cells of the hair follicle absorb it and get stem cells and matrix germinal cells stimulated to multiply and grow to enhance the hair growth.

Step 4: Apply selected hair growth therapeutic agent using modulating VEGF (vascular endothelial growth factor) activity, e.g., modulating VEGF gene expression and/or modulating VEGF protein production and/or activity, to modulate hair growth and/or thickness. The methods can be used to either promote or inhibit hair growth or hair thickness in a subject. Use of VEGR increased perifollicular vascularization promotes hair growth and that expression of VEGF by follicular keratinocytes leads to increased perifollicular vascularization. In addition, it was found that increasing the level of VEGF expression resulted in accelerated hair regrowth and increased hair follicle size, which leads to hair thickening. It was also found that by inhibiting the levels of VEGF, hair growth and hair thickening can be reduced. Applying the VEGF after step 3 insulin application will enhance the activity of VEGF and will lead to rapid hair growth. Insulin itself acts as and enhances the activity of VEGF.

Step 5: Optional step: Absorption enhancers, vasodilators, anti-inflammatory therapeutic agents can be applied if there is an underlying cause affecting the hair loss. This step can be pursued before, during or after the therapeutic agents' application.

### Example 20

Application of insulin hair growth factor with other therapeutic agents such as inhibiting 5α-reductase activity and an ingredient for dilating peripheral blood vessels.

Step 1: Wash the entire scalp, remove oily substances on the balding area with mild shampoo and dry the site of therapeutic intervention thoroughly. It is better to shampoo, dry, and wait for 30 minutes for the water soaked superficial layers of the skin to be dried out completely to enhance the uptake of locally applied hair growth therapeutic agents.

Step 2: Apply oxidant to the balding site with a sprayer or cotton swab. It is an optional step.

Step 3: Apply insulin by Q tip or triangular applicator or soaked cotton swab or by use of a atomizer sprayer. Repeatedly apply insulin as it dries out so that the cells of the hair follicle absorb it and get stem cells and matrix germinal cells stimulated to multiply and grow to enhance the hair growth.

Step 4: Apply selected hair growth promoting therapeutic agent using: a) an ingredient for inhibiting 5 a-reductase activity; b) an ingredient for promoting function of cell activity; and c) an ingredient for dilating peripheral blood vessels

Step 5: Optional step: Absorption enhancers, vasodilators, anti-inflammatory therapeutic agents can be applied if there is an underlying cause affecting the hair loss. This step can be pursued before, during or after the therapeutic agents' application.

### Example 21

Application of insulin hair growth factor with other therapeutic agents such as PGF 2α, and analogues

Step 1: Wash the entire scalp, remove oily substances on the balding area with mild shampoo and dry the site of therapeutic intervention thoroughly. It is better to shampoo, dry, and wait for 30 minutes for the water soaked superficial layers of the skin to be dried out completely to enhance the uptake of locally applied hair growth therapeutic agents.

Step 2: Apply oxidant to the balding site with a sprayer or cotton swab. It is an optional step.

Step 3: Apply insulin by Q tip or triangular applicator or soaked cotton swab or by use of an atomizer sprayer. Repeatedly apply insulin as it dries out so that the cells of the hair follicle absorb it and get stem cells and matrix germinal cells stimulated to multiply and grow to enhance the hair growth.

Apply selected hair growth therapeutic agent using PGF 2α, and analogues. Using these hair growing prostaglandins after pretreatment of the bald site with insulin results in much more rapid hair growth compared to when applied without insulin.

Step 5: Optional step: Absorption enhancers, vasodilators, anti-inflammatory therapeutic agents can be applied if there is an underlying cause affecting the hair loss. This step can be pursued before, during or after the therapeutic agents' application.

### Example 22

Application of insulin hair growth factor with other therapeutic agents such as cholinesterase inhibiting action which has hair growth promoting

Step 1: Wash the entire scalp, remove oily substances on the balding area with mild shampoo and dry the site of therapeutic intervention thoroughly. It is better to shampoo, dry, and wait for 30 minutes for the water soaked superficial layers of the skin to be dried out completely to enhance the uptake of locally applied hair growth therapeutic agents.

Step 2: Apply oxidant to the balding site with a sprayer or cotton swab. It is an optional step.

Step 3: Apply insulin by Q tip or triangular applicator or soaked cotton swab or by use of an atomizer sprayer. Repeatedly apply insulin as it dries out so that the cells of the hair follicle absorb it and get stem cells and matrix germinal cells stimulated to multiply and grow to enhance the hair growth.

Step 4: Then apply selected hair growth promoting therapeutic agent using a compound having a cholinesterase inhibiting action has a hair growth promoting action, and there is provided a hair growth promoting agent containing such a compound having a cholinesterase inhibiting action. Moreover, a compound having an acetylcholinesterase inhibiting action as the cholinesterase inhibiting action is preferable, and in particular donepezil hydrochloride has a good hair growth promoting effect applied as liquid preparation, a cream, an ointment, a plaster or a tape preparation. Moreover, according to the present invention, there is also provided a hair growth promoting method comprising the step of applying donepezil onto the scalp needing hair promoting therapeutic agents after applying insulin to enhance the uptake, activity and spread of this hair growth promoter.

Step 5: Optional step: Absorption enhancers, vasodilators, anti-inflammatory therapeutic agents can be applied if there is an underlying cause affecting the hair loss. This step can be pursued before, during or after the therapeutic agents' application.

### Example 23

Application of insulin hair growth factor with other therapeutic agents such as and use of testosterone and dihyrdotestosterone, potassium channel openers as described above, streptomycin, diphenylhydantoin.

Step 1: Wash the entire scalp, remove oily substances on the balding area with mild shampoo and dry the site of therapeutic intervention thoroughly. It is better to shampoo, dry, and wait for 30 minutes for the water soaked superficial layers of the skin to be dried out completely to enhance the uptake of locally applied hair growth therapeutic agents.

Step 2: Apply oxidant to the balding site with a sprayer or cotton swab. It is an optional step.

Step 3: Apply insulin by Q tip or triangular applicator or soaked cotton swab or by use of an atomizer sprayer. Repeatedly apply insulin as it dries out so that the cells of the hair follicle absorb it and get stem cells and matrix germinal cells stimulated to multiply and grow to enhance the hair growth.

Step 4:: Apply selected hair growth therapeutic agent other than aromatase inhibitors, including testosterone and dihyrdotestosterone on the beard; potassium channel openers as described above, streptomycin, diphenylhydantoin (anticonvulsant drug widely used to control epileptic seizures).

Step 5: Optional step: Absorption enhancers, vasodilators, anti-inflammatory therapeutic agents can be applied if there is an underlying cause affecting the hair loss. This step can be pursued before, during or after the therapeutic agents' application.

### Example 24

Application of insulin growth factor-I (IGF-I) as hair growth factor with other therapeutic agents discussed above.

Step 1: Wash the entire scalp, remove oily substances on the balding area with mild shampoo and dry the site of therapeutic intervention thoroughly. It is better to shampoo, dry, and wait for 30 minutes for the water soaked superficial layers of the skin to be dried out completely to enhance the uptake of locally applied hair growth therapeutic agents.

Step 2: Apply oxidant to the balding site with a sprayer or cotton swab. It is an optional step.

Step 3: Apply insulin growth factor (IGF-1) with sterile Q tip or triangular applicator or cotton swab or by an atomizer spray. Repeatedly apply it as it dries out so that the cells of the hair follicle absorb it completely and get stimulated to multiply and grow to enhance the scalp hair growth.

Step 4: May or may not use other hair growth enhancer discussed here in following the application of IGR-I.

Step 5: Optional step: Absorption enhancers, vasodilators, anti-inflammatory therapeutic agents can be applied if there is an underlying cause affecting the hair loss. This step can be pursued before, during or after the therapeutic agents' application.

### Example 25

Application of insulin growth factor-I (IGF-I) as hair growth factor with other physical and electrical methods to enhance the uptake of therapeutic, pharmaceutical, biochemical and biological agents therapeutic agents including insulin.

Step 1: Wash the entire scalp, remove oily substances on the balding area with mild shampoo and dry the site of therapeutic intervention thoroughly. It is better to shampoo, dry, and wait for 30 minutes for the water soaked superficial layers of the skin to be dried out completely to enhance the uptake of locally applied hair growth therapeutic agents.

Step 2: Apply oxidant to the balding site with a sprayer or cotton swab. It is an optional step.

Step 3: Apply insulin growth factor (IGF-1) with sterile Q tip or triangular applicator or cotton swab or by an atomizer spray. Repeatedly apply it as it dries out so that the cells of the hair follicle absorb it completely and get stimulated to multiply and grow to enhance the scalp hair growth

Step 4: Insulin and IGF-I uptake into hair follicle can be enhanced combined with uptake facilitators such electroporation, iontophoresis, sonophoresis, vibroacoustic, vibration and other physical (heat, magnetic force, radio frequency, microwave, laser lights etc) methods with other appropriate therapeutic, biological and pharmacological hair growth agents combined with insulin. These methods can be used as prophylaxis, to diagnose and to treat scalp hair loss.

Step 5: Optional step: Absorption enhancers, vasodilators, anti-inflammatory therapeutic agents can be applied if there is an underlying cause affecting the hair loss. This step can be pursued before, during or after the therapeutic agents' application.

Numerous modifications and alternative arrangements of steps explained herein may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form function and manner of procedure, assembly and use may be made.

While the preferred embodiment of the present invention has been described, it should be understood that various changes, adaptations and modifications may be made thereto. It should be understood, therefore, that the invention is not limited to details of the illustrated invention.

What is claimed is:

1. A method of increasing the length, thickness, number, and density of scalp hair, comprising the step of administering a therapeutically effective amount of insulin to a person on an area where hair growth is desired.

2. The method of increasing the length, thickness, number, and density of scalp hair according to claim 1 further comprising the step of applying a known hair growth therapeutic agent to said area.

3. The method of increasing the length, thickness, number, and density of hair according to claim 2 wherein said known hair growth therapeutic agent is a pharmaceutical agent.

4. The method of increasing the length, thickness, number, and density of scalp hair according to claim 2 wherein said known hair growth therapeutic agent is a biochemical agent.

5. The method of increasing the length, thickness, number, and density of scalp hair according to claim 2 wherein said known hair growth therapeutic agent is a biological agent.

6. The method of increasing the length, thickness, number, and density of scalp hair according to claim 2 wherein said known hair growth therapeutic agent is minoxidil.

7. The method of increasing the length, thickness, number, and density of scalp hair according to claim 2 wherein said known hair growth therapeutic agent is a finasteride.

8. The method of increasing the length, thickness, number, and density of scalp hair according to claim 2 wherein said known hair growth therapeutic agent is selected from the group consisting of cyclosporine, hexachlorobenzene, danazol, interleukin-4, TGF, TGFa, TGFPI, bFGF, EGF, PDGF, biotin, deferoxamine, prostaglandins, extracts of continus coggygria, PDT, tocotrienols, 5a-reductase inhibitor, antihistaminic, cell activator pantothenyl alcohol, pantothenyl ethyl ether, processed semi-mature soybean, processed semi-mature soybean extract, VEGF, PGF 2α, cholinesterase inhibitors, diphenylhydantoin, and streptomycin.

9. A method of increasing the length, thickness, number, and density of scalp hair, comprising the step of administering a therapeutically effective amount of insulin and IGF-1 to a person on an area where hair growth is desired.

10. The method of increasing the length, thickness, number, and density of scalp hair according to claim 9 further comprising the step of applying a cosmetic coloring agent to scalp hair wherein a desired color is achieved.

11. A method of increasing the length, thickness, number, and density of scalp hair, comprising the step of administering a therapeutically effective amount of IGF-1 to a person on the area where hair growth is desired.

12. The method of increasing the length, thickness, number, and density of scalp hair according to claim 11 further comprising the step of applying a known hair growth therapeutic agent to said area.

13. The method of increasing the length, thickness, number, and density of scalp hair according to claim 11 wherein said known hair growth therapeutic agent is a pharmaceutical agent.

14. The method of increasing the length, thickness, number, and density of scalp hair according to claim 11 wherein said known hair growth therapeutic agent is selected from the group consisting of cyclosporine, hexachlorobenzene, danazol, interleukin-4, TGF, TGFa, TGFPI, bFGF, EGF, PDGF, biotin, deferoxamine, prostaglandins, extracts of continus coggygria, PDT, tocotrienols, 5a-reductase inhibitor, antihistaminic, cell activator pantothenyl alcohol, pantothenyl ethyl ether, processed semi-mature soybean, processed semi-mature soybean extract, VEGF, PGF 2α, cholinesterase inhibitors, diphenylhydantoin, and streptomycin

15. The method of increasing the length, thickness, number, and density of scalp hair according to claim 9 further comprising the step of applying a known hair growth therapeutic agent to said area and then enhance their uptake and therapeutic activity by using into hair follicle can be enhanced combined with uptake facilitators such electroporation, iontophoresis, sonophoresis, vibroacoustic, vibration and other physical (heat, magnetic force, radio frequency, microwave, laser lights etc) methods with other appropriate therapeutic, biological and pharmacological hair growth agents combined with insulin. These methods can be used as prophylaxis, to diagnose and to treat scalp hair loss.
